(19) United States
(12) Patent Application Publication
(10) Pub. No.: US 2024/0352450 A1
(43) Pub. Date: Oct. 24, 2024

(54) Systems and Methods to Determine Nucleic Acid Conformations and Uses Thereof

(71) Applicant: The Board of Trustees of the Leland Stanford Junior University, Stanford, CA (US)

(72) Inventors: Hannah K. Wayment-Steele, Boston, MA (US); Eesha Sharma, Stanford, CA (US); Rhiju Das, Palo Alto, CA (US); William J. Greenleaf, Menlo Park, CA (US); Anthony Ho, Seattle, WA (US); Winston Becker, Stanford, CA (US); Yuxi Ke, San Carlos, CA (US)

(73) Assignee: The Board of Trustees of the Leland Stanford Junior University, Stanford, CA (US)

(21) Appl. No.: 18/687,249

(22) PCT Filed: Aug. 29, 2022
(86) PCT No.: PCT/US2022/075607

§ 371 Date: Feb. 27, 2024

Related U.S. Application Data
(60) Provisional application No. 63/245,744, filed on Sep. 17, 2021.
(60) Provisional application No. 63/238,055, filed on Aug. 27, 2021.

(51) Int. Cl. C12N 15/10

(52) U.S. Cl. CPC C12N 15/1065

(57) ABSTRACT

Embodiments herein describe systems and methods to determine nucleic acid thermodynamics and uses thereof. Many embodiments utilize a sequencing chip, such as an Illumina flow cell as a high-throughput platform for performing massively parallel melt curve determination. In many embodiments, nucleic acid molecules possessing a region that forms a secondary structure are affixed to a sequencing chip and hybridized with one or more labeled oligonucleotides. As the secondary structure denatures, changes in fluorescence can be measured to determine a melt curve of specific sequences.

### CROSS-REFERENCE TO RELATED APPLICATIONS

The current application claims priority to U.S. Provisional Patent Application No. 63/238,055, filed Aug. 27, 2021 and U.S. Provisional Patent Application No. 63/245,744, filed Sep. 17, 2021; the disclosures of which are hereby incorporated by reference in their entireties.

### STATEMENT REGARDING FEDERALLY SPONSORED RESEARCH OR DEVELOPMENT

This invention was made with Government support under contracts GM122579 and HG007735 awarded by the National Institutes of Health. The Government has certain rights in the invention.

### FIELD OF THE INVENTION

The present invention relates to nucleic acid conformations. More specifically, the present invention relates to systems and methods for high throughput determination of the nucleic acid conformations from nucleic acid sequences.

### BACKGROUND

Base-pairing in DNA and RNA molecules underlies many critical processes in biology, including signaling, viral replication and packaging, catalysis, structure of noncoding RNAs, as well as in biotechnology, such as design of improved constructs and protocols for PCR amplification. (See e.g., Soukup, G. A. and Breaker, R. R. (2000) Allosteric nucleic acid catalysts. Curr. Opin. Struct. Biol., 10, 318-325; Amaral, P. P., Dinger, M. E., Mercer, T. R. and Mattick, J. S. (2008) The eukaryotic genome as an RNA machine. Science, 319, 1787-1789; and Tian, S., Yesselman, J. D., Cordero, P. and Das, R. (2015) Primerize: automated primer assembly for transcribing non-coding RNA domains. Nucleic Acids Res, 43, W522-526; the disclosures of which are hereby incorporated by reference in their entireties.) Numerous algorithms have been developed to predict DNA and RNA secondary structure thermodynamics, many of which make use of parameters inferred from optical melting experiments on a handful of constructs. (See e.g., Lorenz, R., Bernhart, S. H., Honer Zu Siederdissen, C., Tafer, H., Flamm, C., Stadler, P. F. and Hofacker, I. L. (2011) ViennaRNA Package 2.0. Algorithms Mol Biol, 6, 26; Zadeh, J. N., Steenberg, C. D., Bois, J. S., Wolfe, B. R., Pierce, M. B., Khan, A. R., Dirks, R. M. and Pierce, N. A. (2011) NUPACK: Analysis and design of nucleic acid systems. J Comput Chem, 32, 170-173; Reuter, J. S. and Mathews, D. H. (2010) RNAstructure: software for RNA secondary structure prediction and analysis. BMC Bioinformatics, 11, 129; and Xia, T., SantaLucia, J., Jr., Burkard, M. E., Kierzek, R., Schroeder, S. J., Jiao, X., Cox, C. and Turner, D. H. (1998) Thermodynamic parameters for an expanded nearest-neighbor model for formation of RNA duplexes with Watson-Crick base pairs. Biochemistry, 37, 14719-14735; the disclosures of which are hereby incorporated by reference in their entireties.) Recent work with more high throughput readouts of nucleic acid structure have demonstrated that algorithms based on these optical melting experiments perform poorly at predicting experimental observables such as RNA-protein binding constants and RNA structure mapping experiments. (See e.g., Becker, W. R., Jarmoskaite, I., Kappel, K., Vaidyanathan, P. P., Denny, S. K., Das, R., Greenleaf, W. J. and Herschlag, D. (2019) Quantitative high-throughput tests of ubiquitous RNA secondary structure prediction algorithms via RNA/protein binding. bioRxiv, 571588; and Wayment-Steele, H. K., Kladwang, W., Participants, E. and Das, R. (2020) RNA secondary structure packages ranked and improved by high-throughput experiments. bioRxiv. 10.1101/2020.05.29.124511, pre-print: not peer-reviewed; the disclosures of which are hereby incorporated by reference in their entireties.) A major bottleneck limiting prior model development is the throughput available to methods that characterize DNA and RNA duplexes one-by-one.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to a method for measuring nucleic acid thermodynamics, including obtaining a library of nucleic acid molecules, where each molecule in the library includes a first oligonucleotide complementary region, a second oligonucleotide complementary region, and a query region, where the query region includes a sequence of interest to calculate thermodynamics of a secondary structure formed within the query region, where the first oligonucleotide complementary region is located 5′ of the query region and the second oligonucleotide complementary region is located 3′ of the query region, affixing the library of nucleic acid molecules to a nucleic acid sequencing chip, hybridizing a first oligonucleotide to the first oligonucleotide complementary region and a second oligonucleotide to the second oligonucleotide complementary region of each molecule in the library of nucleic acid molecules affixed to the sequencing chip, where the first oligonucleotide includes a first tag at its 5′ end and the second oligonucleotide includes a second tag at its 3′ end, where the first tag and the second tag are capable of interacting when within a specified distance each other, and where a structure formed in the query region brings the first tag and the second tag within the specified distance, altering a parameter of the nucleic acid sequencing chip, where a change in the parameter affects a structure formed in the query region, and measuring a signal emitted from at least one of the first tag and the second tag as the parameter changes.

In some aspects, the techniques described herein relate to a method, where the parameter is selected from pH, salt composition, salt concentration, buffer composition, buffer concentration, organic molecule composition, organic molecule concentration, temperature, and combinations thereof.

In some aspects, the techniques described herein relate to a method, where the parameter is salt composition.

In some aspects, the techniques described herein relate to a method, where the salt within the salt composition is selected from sodium chloride and potassium chloride.

In some aspects, the techniques described herein relate to a method, where the parameter is buffer composition.

In some aspects, the techniques described herein relate to a method, where the buffer within the buffer composition is selected from sodium phosphate, sodium bisphosphate, sodium carbonate, sodium bicarbonate, potassium phosphate, potassium bisphosphate, potassium carbonate, potassium bicarbonate, sodium acetate, and potassium acetate.

In some aspects, the techniques described herein relate to a method, where the parameter is temperature.

In some aspects, the techniques described herein relate to a method, where the temperature ramps from approximately 4° C. to 90° C.

In some aspects, the techniques described herein relate to a method, where the first tag or the second tag is a fluorophore.

In some aspects, the techniques described herein relate to a method, where the first tag and the second tag are fluorophores.

In some aspects, the techniques described herein relate to a method, where the emission wavelength of the first tag is the excitation wavelength of the second tag.

In some aspects, the techniques described herein relate to a method, where the emission wavelength of the second tag is the excitation wavelength of the first tag.

In some aspects, the techniques described herein relate to a method, where the first tag is a fluorophore and the second tag is a quencher.

In some aspects, the techniques described herein relate to a method, where the emission wavelength of the first tag is an absorbance wavelength of the second tag.

In some aspects, the techniques described herein relate to a method, where the first tag is a quencher and the second tag is a fluorophore.

In some aspects, the techniques described herein relate to a method, where the emission wavelength of the second tag is an absorbance wavelength of the first tag.

In some aspects, the techniques described herein relate to a method, where the sequencing chip is an Illumina flow cell.

In some aspects, the techniques described herein relate to a method, further including sequencing each molecule in the affixed library of nucleic acid molecules.

In some aspects, the techniques described herein relate to a method, where sequencing identifies a coordinate of each molecule in the affixed library of nucleic acid molecules.

In some aspects, the techniques described herein relate to a method, further including transcribing each molecule in the affixed library of nucleic acid molecules into RNA, where hybridizing a first oligonucleotide hybridizes the first oligonucleotide to the RNA.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where the nucleic acid molecules are selected from DNA, RNA, LNA, and combinations thereof.

In some aspects, the techniques described herein relate to a method for predicting nucleic acid thermodynamics, including obtaining high-throughput measurements of nucleic acid thermodynamics, training a machine learning model based on the thermodynamics of specific sequences in the high-throughput measurements, and predicting thermodynamics of a query sequencing using the machine learning model.

In some aspects, the techniques described herein relate to a method, where obtaining high-throughput measurements includes obtaining a library of nucleic acid molecules, where each molecule in the library includes a first oligonucleotide complementary region, a second oligonucleotide complementary region, and a query region, where the query region includes a sequence of interest to calculate thermodynamics of a secondary structure formed within the query region, where the first oligonucleotide complementary region is located 5′ of the query region and the second oligonucleotide complementary region is located 3′ of the query region, affixing the library of nucleic acid molecules to a nucleic acid sequencing chip, hybridizing a first oligonucleotide to the first oligonucleotide complementary region and a second oligonucleotide to the second oligonucleotide complementary region of each molecule in the library of nucleic acid molecules affixed to the sequencing chip, where the first oligonucleotide includes a first tag at its 5′ end and the second oligonucleotide includes a second tag at its 3′ end, where the first tag and the second tag are capable of interacting when within a specified distance each other, and where a structure formed in the query region brings the first tag and the second tag within the specified distance, altering a parameter of the nucleic acid sequencing chip, where a change in the parameter affects a structure formed in the query region, and measuring a signal emitted from at least one of the first tag and the second tag as the parameter changes.

In some aspects, the techniques described herein relate to a method, where the parameter is selected from pH, salt composition, salt concentration, buffer composition, buffer concentration, organic molecule composition, organic molecule concentration, temperature, and combinations thereof.

In some aspects, the techniques described herein relate to a method, where the parameter is salt composition.

In some aspects, the techniques described herein relate to a method, where the salt within the salt composition is selected from sodium chloride and potassium chloride.

In some aspects, the techniques described herein relate to a method, where the parameter is buffer composition.

In some aspects, the techniques described herein relate to a method, where the buffer within the buffer composition is selected from sodium phosphate, sodium bisphosphate, sodium carbonate, sodium bicarbonate, potassium phosphate, potassium bisphosphate, potassium carbonate, potassium bicarbonate, sodium acetate, and potassium acetate.

In some aspects, the techniques described herein relate to a method, where the parameter is temperature.

In some aspects, the techniques described herein relate to a method, where the temperature ramps from approximately 4° C. to 90° C.

In some aspects, the techniques described herein relate to a method, where the first tag or the second tag is a fluorophore.

In some aspects, the techniques described herein relate to a method, where the first tag and the second tag are fluorophores.

In some aspects, the techniques described herein relate to a method, where the emission wavelength of the first tag is the excitation wavelength of the second tag.

In some aspects, the techniques described herein relate to a method, where the emission wavelength of the second tag is the excitation wavelength of the first tag.

In some aspects, the techniques described herein relate to a method, where the first tag is a fluorophore and the second tag is a quencher.

In some aspects, the techniques described herein relate to a method, where the emission wavelength of the first tag is an absorbance wavelength of the second tag.

In some aspects, the techniques described herein relate to a method, where the first tag is a quencher and the second tag is a fluorophore.

In some aspects, the techniques described herein relate to a method, where the emission wavelength of the second tag is an absorbance wavelength of the first tag.

In some aspects, the techniques described herein relate to a method, where the sequencing chip is an Illumina flow cell.

In some aspects, the techniques described herein relate to a method, further including sequencing each molecule in the affixed library of nucleic acid molecules.

In some aspects, the techniques described herein relate to a method, where sequencing identifies a coordinate of each molecule in the affixed library of nucleic acid molecules.

In some aspects, the techniques described herein relate to a method, further including transcribing each molecule in the affixed library of nucleic acid molecules into RNA, where hybridizing a first oligonucleotide hybridizes the first oligonucleotide to the RNA.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where the nucleic acid molecules are selected from DNA, RNA, LNA, and combinations thereof.

In some aspects, the techniques described herein relate to a method for measuring interactions between a nucleic acid and another molecule including obtaining a library of nucleic acid molecules, where each molecule in the library includes a query region, where the query region includes a sequence of interest to determine an interaction between the query region and another molecule and a first tag affixed to the query region, affixing the library of nucleic acid molecules to a nucleic acid sequencing chip, introducing a query molecule to the nucleic acid sequencing chip to allow an interaction to form between the query region of at least one nucleic acid molecule in the library of nucleic acid molecules and the query molecule, where the query molecule includes a second tag, and where an interaction between the query region of the at least one nucleic acid molecule and the query molecule brings the first tag and the second tag within a specified distance of each other, where the specified distance allows the first tag and second tag to interact, altering a parameter of the nucleic acid sequencing chip, where a change in the parameter affects an interaction between a query region and a query molecule, and measuring a signal emitted from at least one of the first tag and the second tag as the parameter changes.

In some aspects, the techniques described herein relate to a method, where the parameter is selected from pH, salt composition, salt concentration, buffer composition, buffer concentration, organic molecule composition, organic molecule concentration, temperature, and combinations thereof.

In some aspects, the techniques described herein relate to a method, where the parameter is salt composition.

In some aspects, the techniques described herein relate to a method, where the salt within the salt composition is selected from sodium chloride and potassium chloride.

In some aspects, the techniques described herein relate to a method, where the parameter is buffer composition.

In some aspects, the techniques described herein relate to a method, where the buffer within the buffer composition is selected from sodium phosphate, sodium bisphosphate, sodium carbonate, sodium bicarbonate, potassium phosphate, potassium bisphosphate, potassium carbonate, potassium bicarbonate, sodium acetate, and potassium acetate.

In some aspects, the techniques described herein relate to a method, where the parameter is temperature.

In some aspects, the techniques described herein relate to a method, where the temperature ramps from approximately 4° C. to 90° C.

In some aspects, the techniques described herein relate to a method, where the first tag or the second tag is a fluorophore.

In some aspects, the techniques described herein relate to a method, where the first tag and the second tag are fluorophores.

In some aspects, the techniques described herein relate to a method, where the emission wavelength of the first tag is the excitation wavelength of the second tag.

In some aspects, the techniques described herein relate to a method, where the emission wavelength of the second tag is the excitation wavelength of the first tag.

In some aspects, the techniques described herein relate to a method, where the first tag is a fluorophore and the second tag is a quencher.

In some aspects, the techniques described herein relate to a method, where the emission wavelength of the first tag is an absorbance wavelength of the second tag.

In some aspects, the techniques described herein relate to a method, where the first tag is a quencher and the second tag is a fluorophore.

In some aspects, the techniques described herein relate to a method, where the emission wavelength of the second tag is an absorbance wavelength of the first tag.

In some aspects, the techniques described herein relate to a method, where the sequencing chip is an Illumina flow cell.

In some aspects, the techniques described herein relate to a method, further including sequencing each molecule in the affixed library of nucleic acid molecules.

In some aspects, the techniques described herein relate to a method, where sequencing identifies a coordinate of each molecule in the affixed library of nucleic acid molecules.

In some aspects, the techniques described herein relate to a method, further including transcribing each molecule in the affixed library of nucleic acid molecules into RNA, where hybridizing a first oligonucleotide hybridizes the first oligonucleotide to the RNA.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where measuring the signal includes imaging the sequencing chip, increasing temperature on the sequencing chip, and reimaging the sequencing chip.

In some aspects, the techniques described herein relate to a method, where measuring the signal further includes reincreasing temperature on the sequencing chip, and reimaging the sequencing chip.

In some aspects, the techniques described herein relate to a method, where the nucleic acid molecules are selected from DNA, RNA, LNA, and combinations thereof.

In some aspects, the techniques described herein relate to a method, where the query molecule is selected from a nucleic acid, a protein, a peptide, a carbohydrate, an organic compound, and combinations thereof.

In some aspects, the techniques described herein relate to a method for determining composition of a complex mixture, including obtaining a library of nucleic acid molecules affixed to a sequencing chip, where each molecule in the library includes an aptamer region, a self-complementary region, a first complementary region, and a second complementary region, where the aptamer region is flanked by the self-complementary region and the second complementary region, and the first complementary region is located adjacent to the second complementary region, and where the self-complementary region is complementary to the second complementary region, hybridizing a first oligonucleotide to the first complementary region and a second oligonucleotide to the second complementary region of each molecule in the library of nucleic acid molecules, where the first oligonucleotide includes a first tag and the second oligonucleotide includes a second tag, where the first tag and the second tag are capable of interacting when within a specified distance each other, and where hybridization of the first oligonucleotide to the first complementary region and the second oligonucleotide to the second complementary region brings the first tag and second tag within the specified distance, introducing a sample to the sequencing chip, where the sample includes small molecules of interest, where an interaction between a small molecule in the sample to an aptamer region causes a conformational change in a nucleic acid molecule which displaces the second oligonucleotide from the second complementary region and allows the self-complementary region to bind to the second complementary region, and measuring a signal emitted from the first tag as an indicator of an interaction between an aptamer region and a small molecule interaction.

In some aspects, the techniques described herein relate to a method, where the sample is selected from a biological sample and an environmental sample.

In some aspects, the techniques described herein relate to a method, where the first tag or the second tag is a fluorophore.

In some aspects, the techniques described herein relate to a method, where the first tag and the second tag are fluorophores.

In some aspects, the techniques described herein relate to a method, where the emission wavelength of the first tag is the excitation wavelength of the second tag.

In some aspects, the techniques described herein relate to a method, where the emission wavelength of the second tag is the excitation wavelength of the first tag.

In some aspects, the techniques described herein relate to a method, where the first tag is a fluorophore and the second tag is a quencher.

In some aspects, the techniques described herein relate to a method, where the emission wavelength of the first tag is an absorbance wavelength of the second tag.

In some aspects, the techniques described herein relate to a method, where the first tag is a quencher and the second tag is a fluorophore.

In some aspects, the techniques described herein relate to a method, where the emission wavelength of the second tag is an absorbance wavelength of the first tag.

In some aspects, the techniques described herein relate to a method, where the sequencing chip is an Illumina flow cell.

In some aspects, the techniques described herein relate to a method, further including sequencing each molecule in the affixed library of nucleic acid molecules.

In some aspects, the techniques described herein relate to a method, where sequencing identifies a coordinate of each molecule in the affixed library of nucleic acid molecules.

In some aspects, the techniques described herein relate to a method, where the nucleic acid molecules are selected from DNA, RNA, LNA, and combinations thereof.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates an exemplary schematic of a nucleic acid molecule to identify intramolecular thermodynamics and/or conformations in accordance with various embodiments of the invention.

FIG. 1B illustrates exemplary secondary structures of nucleic acid molecules in accordance with various embodiments of the invention.

FIG. 1C illustrate an exemplary schematic of a quenching-based assay to measure nucleic acid thermodynamics in accordance with various embodiments of the invention.

FIG. 1D illustrates a method for high-throughput screening of nucleic acid molecules in accordance with various embodiments of the invention.

FIG. 2A illustrates an exemplary schematic of a nucleic acid molecule to identify intermolecular thermodynamics and/or conformations in accordance with various embodiments of the invention.

FIG. 2B illustrates a method for high-throughput screening of nucleic acid molecules in accordance with various embodiments of the invention.

FIG. 2C illustrates an exemplary schematic of a nucleic acid molecule for chemical sensing in accordance with various embodiments of the invention.

FIG. 2D illustrates a schematic of a quenching-based assay for chemical sensing in accordance with various embodiments of the invention.

FIGS. 3A-3B illustrate exemplary data for normalizing fluorescence for thermal effects in accordance with various embodiments of the invention.

FIGS. 4A-4D illustrate exemplary data for nucleotide proximity effects in accordance with various embodiments of the invention.

FIG. 5A illustrates a flow chart of an exemplary method to train a machine learning model in accordance with various embodiments of the invention.

FIG. 5B illustrates a flow chart of an exemplary method to determine nucleic acid conformations in accordance with various embodiments of the invention.

FIG. 5C illustrates a block diagram of components of a processing system in a computing device that can be used to determine nucleic acid conformations in accordance with various embodiments of the invention.

FIG. 5D illustrates a network diagram of a distributed system to determine nucleic acid conformations in accordance with various embodiments of the invention.

FIGS. 6A-6E illustrate exemplary embodiments to determine hairpin stability in accordance with various embodiments of the invention. FIG. 6A: Two versions of each construct are characterized: one with no quench-oligo bound (Control) to monitor the max fluorescence at each temperature, and one with a quench-labeled oligomer annealed. Fraction unfolded is calculated as the fluorescence of the quench version divided by the fluorescence of the control version, and resulting parameters are inferred by fitting the probability of the closing base pair forming. FIG. 6B: Variation in fluorescence based on the first two nucleotides of the variable region in the library motivated the need for a control fluorescence variant. FIG. 6C: Unstructured controls, as well as controls containing a stem but varying an upstream (FIG. 6D) or downstream unstructured linker length (FIG. 6E) demonstrated that constructs reach a maximum fluorescence at ˜16 nucleotides.

FIGS. 7A-7D illustrate exemplary data for quality of thermodynamic fits depending on temperature range in accordance with various embodiments of the invention. FIG. 7A: Fit melting temperatures (Tm) and enthalpy (DH) for datapoints excluding Tm and DH outliers. Example data from starred locations are depicted in bottom row. Black line represents unstructured controls. FIG. 7B: Datapoints with DH std. err<5 kcal/mol. FIG. 7C: Datapoints with Tm std. err<10 K. FIG. 7D: Dependence of standard error on number of clusters and NUPACK-predicted DG (37° C.).

FIGS. 8A-8D illustrate exemplary data for nearest-neighbor model providing best test set predictions in accordance with various embodiments of the invention. FIG. 8A: 643 constructs from the Watson-Crick series were selected with error<1.0 kcal/mol. DG of each construct was predicted from a linear model of features of varying complexity in a k-fold cross-validation scheme. This resulted in the nearest-neighbor model having the best RMSE and BIC. FIG. 8B: The resulting parameters from one of the k-fold ridge regression models correlates closely with NUPACK parameters. FIG. 8C: An expanded dataset of 3121 constructs that also include G-T mismatches shows the best RMSE with the triplet-neighbor model, though the nearest-neighbor model has the best BIC. FIG. 8D: Comparing parameters to NUPACK parameters again show the fit data agree with the NUPACK parameters.

FIG. 9 illustrates exemplary data showing sample melt curves of structured molecules in accordance with various embodiments of the invention.

FIGS. 10A-10E illustrate exemplary data of highly multiplexed chemical sensing on a sequencing chip in accordance with various embodiments of the invention.

FIGS. 11A-11C illustrate exemplary data of machine learning model performance in accordance with various embodiments of the invention.

### DETAILED DESCRIPTION

Turning now to the drawings, systems and methods to determine nucleic acid conformations and uses thereof are provided. Ribonucleic acid molecules (RNA molecules or RNAs) are known to form various secondary structures, such as hairpins, loops, and junctions. Many of these structures can improve the stability of an RNA molecule. Many embodiments herein describe high throughput methodologies to determine stability of these secondary structure motifs. Certain embodiments utilize a platform capable of massively parallel fluorescence measurements to identify equilibrium of nucleic acid hairpin formation to make quantitative measurements of the thermodynamics and/or conformations of the nucleic acid secondary structure. Further embodiments allow for predictive models of RNA stability in different contexts, such as different ionic concentrations, modified nucleotides, and molecular crowder buffer conditions.

### Assessment of Nucleic Acid Structure Conformations

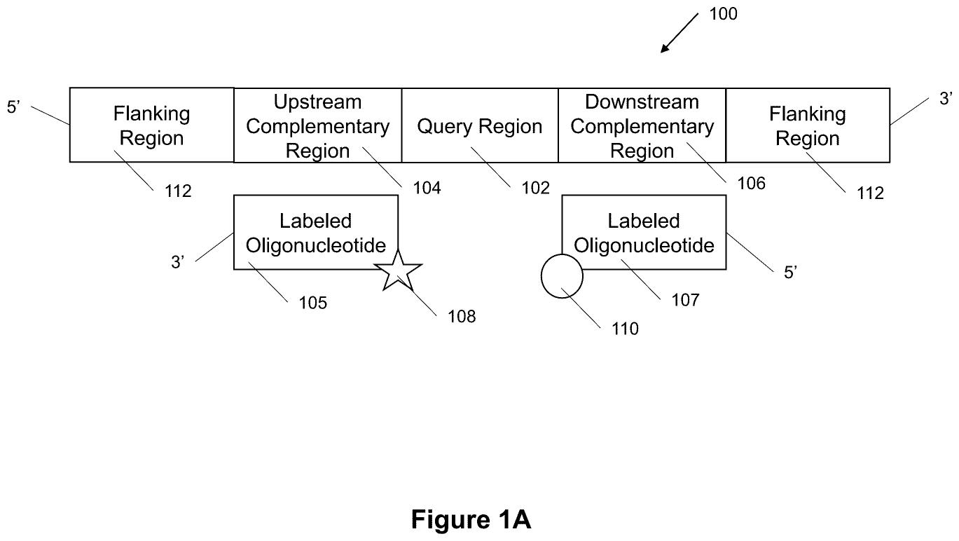

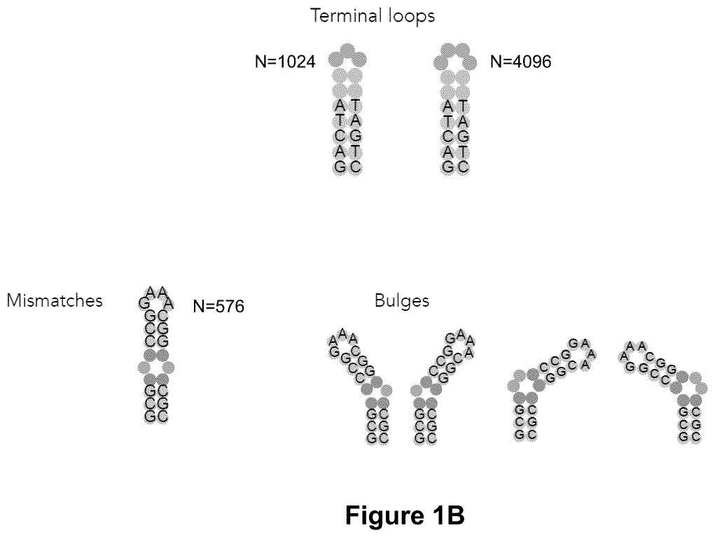

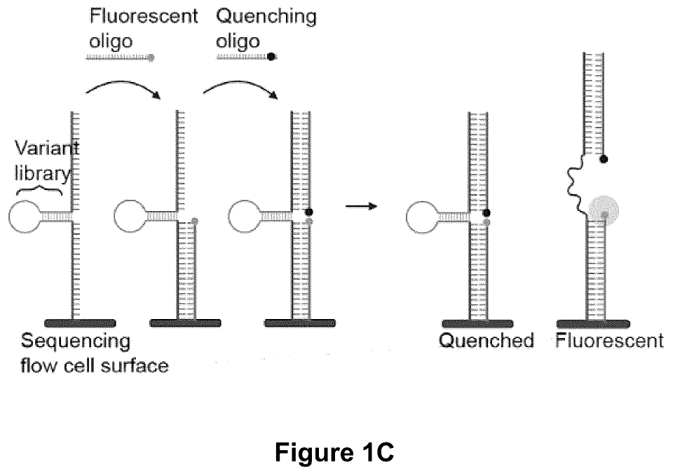

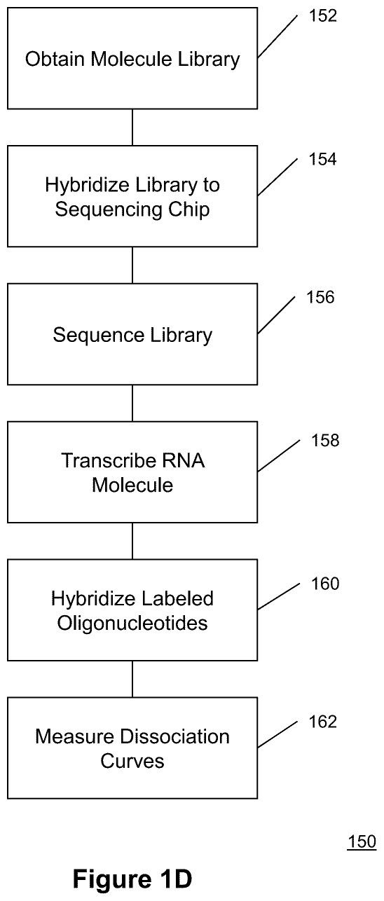

Many embodiments are capable of assessing the thermodynamics and/or structural conformations of nucleic acid structures formed within a single stranded nucleic acid molecule, (e.g., DNA, RNA, and/or LNA). FIG. 1A illustrates an exemplary molecule 100 in accordance with various embodiments to assess structure formed by a nucleic acid molecule. In many embodiments, molecule 100 is a single stranded molecule. The molecule 100 comprises a 5′ end and a 3′ end and a query region 102. In many embodiments the query region 102 comprises a sequence that forms a secondary structure, such as a hairpin, mismatch, bulge, loop, and/or any other secondary structure. In some embodiments, query region 102 comprises a random sequence and/or a sequence with unknown ability to form a secondary structure.

FIG. 1B illustrates a few examples of secondary structures, including terminal loops, mismatches, and bulges, which can be formed by a query region 102. In certain embodiments, a query region 102 comprises between at least 2 nucleotides, including 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 22 nucleotides, 24 nucleotides, 26 nucleotides, 28 nucleotides, 30 nucleotides, 32 nucleotides, 34 nucleotides, 36 nucleotides, 38 nucleotides, 40 nucleotides, 42 nucleotides, 44 nucleotides, 46 nucleotides, 48 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, or a greater number of nucleotides.

Returning to FIG. 1A, additional embodiments include one or more complementary regions. Complementary regions can be an upstream complementary region 104 located at the 5′ end of a query region 102 or a downstream complementary region 106 located at the 3′ end of a query region 102. Upstream complementary region 104 and/or downstream complementary region 106 in accordance with various embodiments allow for binding of a labeled oligonucleotide 105, 107. In many embodiments, an upstream labeled oligonucleotide 105 is complementary to (e.g., reverse complement of or otherwise able to bind to) the upstream complementary region 104, while a downstream labeled oligonucleotide 107 is complementary to (e.g., reverse complement of or otherwise able to bind to) the downstream complementary region 106. Labeled oligonucleotides 105, 107 can comprise a molecular tag 108, 110 on the molecule. In many embodiments tags 108, 110 are capable of interacting when within a specified distance. Such interactions can include amplification, positive interference, negative interference, suppression, or other interaction. Such interactions can augment a signal, emission, or other property of the tags 108, 110. In various embodiments, an upstream labeled oligonucleotide 105 can have a tag 108 at its 5′ end, while a downstream labeled oligonucleotide 107 can have a tag 110 at its 3′ end. In such an arrangement, tags 108, 110 can be brought within a specified distance (e.g., the distance of interaction) by a structure formed by the query region 102.

In many embodiments, tags 108, 110 are selected from fluorophores, quenchers, and/or any other relevant or applicable tag. Many fluorophores are known, including (but not limited to) fluorescein, fluorescein isothiocyanate (FITC), Cy3, Cy5, rhodamine, rhodopsin, Alexafluor488, Alexafluor647, and/or any other known fluorophore known in the art. Interactions between tags 108, 110 can include fluorescence resonance energy transfer (FRET), where an emission wavelength of one fluorophore is an excitation wavelength of another fluorophore, or quenching, where a tag absorbs light at the emission wavelength of a fluorophore.

It should be noted that upstream complementary region 104 and downstream complementary region 106 can also be referred to as a “first complementary region” or “second complementary region” as ambivalent references to position (e.g., a first complementary region can refer to either the upstream complementary region 104 or downstream complementary region 106). Similarly, upstream labeled oligonucleotide 105 and downstream labeled oligonucleotide 107 can also be referred to as a “first labeled oligonucleotide” or “second labeled oligonucleotide” as ambivalent references to position.

FIG. 1C illustrates a schematic of a fluorescence-quenching process as described herein. As illustrated in FIG. 1C, a structured nucleic acid is illustrated, which can then be hybridized with a fluorophore-labeled oligonucleotide and a quencher-labeled oligonucleotide. As a condition within the environment changes, (e.g., increasing temperature, pH, salt composition, salt concentration, etc.), the structure may dissociate. The dissociation can cause a fluorophore and quencher out of proximity to each other, thus allowing the fluorescence to reoccur.

Returning to FIG. 1C, in various embodiments, the molecules in the library comprise one or more flanking sequences 112 located 5′ of an upstream complementary region 104 and/or 3′ of a downstream complementary region 108. Such flanking sequences 112 can be selected from one or more of sequencing primer sequences, adapter sequences, barcoding sequences, and/or any other relevant sequence. In certain embodiments, sequencing primer sequences and/or adapter sequences are unique or relevant a particular sequencing platform (e.g., Illumina, Ion Torrent, etc.). One of skill in the art will understand specific sequences can be used for hybridization, amplification, and/or any other necessary property to build a library of molecules for use as a flanking sequence 112.

Various embodiments have flanking sequences that allow the molecules to hybridize to a sequencing chip, such as an Illumina flow cell, and perform cluster generation. Exemplary Illumina flow cells include flow cells for a MiSeq, HiSeq, iSeq, MiniSeq, NextSeq, NovaSeq, and/or any other sequencing instrument using such flow cells.

Many embodiments can utilize molecules, such as molecule 100 in to screen for nucleic acid thermodynamics and/or conformation. Turning to FIG. 1D, a method 150 for high throughput thermodynamic and/or conformation screening is illustrated. At 152, many embodiments obtain a library of molecules, where each molecule in the library comprises a query sequence of interest. A library in various embodiments includes any number of molecules, such as 1 molecule, 2 molecules, 3 molecules, 5 molecules, 10 molecules, 25 molecules, 50 molecules, 75 molecules, 100 molecules, 150 molecules, 200 molecules, 250 molecules, 300 molecules, 350 molecules, 400 molecules, 450 molecules, 500 molecules, 750 molecules, 800 molecules, 850 molecules, 900 molecules, 950 molecules, 800 molecules, 900 molecules, 1000 molecules, 1250 molecules, 1500 molecules, 1750 molecules, 2000 molecules, 2500 molecules, 5000 molecules, 10,000 molecules, 15,000 molecules, 20,000 molecules, 25,000 molecules, 50,000 molecules, 100,000 molecules, 250,000 molecules, 500,000 molecules, 1,000,000 molecules, 2,000,000 molecules, 5,000,000 molecules, 10,000,000 molecules, or more molecules. In many embodiments, the query sequence of interest is designed to form a secondary structure within a query region, such as described herein and/or known in the art. Certain embodiments possess a structure similar to the exemplary molecule illustrated in FIG. 1A (e.g., molecule 100). In many embodiments, the molecule is a nucleic acid molecule, such as a DNA molecule, an RNA molecule, and LNA molecule, and/or a combination of multiple types of nucleic acid molecules. In many embodiments, the full length of each molecule in the library has between 10 and 200 nucleotides in length, such as 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 125 nucleotides, 130 nucleotides, 140 nucleotides, 150 nucleotides, 160 nucleotides, 170 nucleotides, 175 nucleotides, 180 nucleotides, 190 nucleotides, or 200 nucleotides. In such embodiments, the thermodynamics or conformation of the nucleic acid molecules, including oligonucleotides, small RNAs, primers (e.g., for PCR), or any other molecule can be measured by a structure formed within a query region.

Many embodiments obtain the library as DNA molecule analogs of RNA and/or LNA sequences, while certain embodiments obtain the library as RNA molecules and/or LNA molecules. Further embodiments synthesize the molecules directly as DNA, RNA and/or LNA via any applicable means, such as transcription, polymerization, and/or ordering such sequences from third party sources or vendors. Various embodiments amplify the molecular library to increase the copy number of sequences, through means, such as PCR or other known methodologies.

At 154, various embodiments hybridize the molecular library to a sequencing chip or flow cell. Various sequencing platforms operate differently, such that certain molecules possess a sequencing chip (e.g., Ion Torrent, Roche 454), while others possess a flow cell (e.g., Illumina). Some embodiments utilize adapter sequences within the molecules to allow the molecules to hybridize to the sequencing chip or flow cell. Many embodiments utilize an Illumina flow cell, such as a flow cell from a Genome Analyzer, MiSeq, HiSeq, HiScan, iSeq, MiniSeq, NextSeq, NovaSeq and/or any other Illumina sequencing platform. Once hybridized to an Illumina flow cell, certain embodiments generate clusters on the Illumina flow cell. Such embodiments follow known methods of amplifying molecules on a flow cell. On other platforms, similar processes can be undertaken to generate molecules attached to a sequencing chip-such processes are specific to the sequencing platform and can be identified in manuals or other literature specific to such platforms.

Certain embodiments sequence the molecules at 156. Such methods are known in the art. In the situation of many sequencing platforms, including Illumina platforms, sequencing reveals the location and/or coordinates of specific sequences, which correlate to individual molecules within the library. Such sequences are identified by the sequencing process.

Embodiments measuring RNA thermodynamics and/or conformation transcribe an RNA and/or LNA molecule anchored to the flow cell or sequencing chip at 158. Such embodiments can generate the RNA via methods, such as those described in She, R., Chakravarty, A. K., Layton, C. J., Chircus, L. M., Andreasson, J. O., Damaraju, N., McMahon, P. L., Buenrostro, J. D., Jarosz, D. F. and Greenleaf, W. J. (2017) Comprehensive and quantitative mapping of RNA-protein interactions across a transcribed eukaryotic genome. Proc Natl Acad Sci USA, 114, 3619-3624; the disclosure of which is hereby incorporated by reference in its entirety. LNA or other forms of nucleic acid molecules can be generated with similarly applicable methods. However, measuring DNA thermodynamics and/or conformation, such methods may not be necessary, if molecules are already in DNA form.

At 160, many embodiments hybridize one or more labeled oligonucleotides (e.g., labeled oligonucleotides 105, 107, FIG. 1A) to the molecules. Some embodiments allow for hybridization of two labeled oligonucleotides, such that in a structured form (e.g., when forming a hairpin) brings the tags (e.g., tags 108, 110, FIG. 1A) in proximity to each other—e.g., at a specified distance such that the tags can interact (e.g., via FRET, quenching, and/or any other form of interaction described herein). While some molecules act as a control and only allow for hybridization of a fluorescently labeled oligonucleotide. Hybridization in many embodiments comprises melting the nucleic acid and annealing the labeled oligonucleotides to the molecules on the sequencing chip. In some embodiments, melting the nucleic acid involves heating the nucleic acids followed and reducing the temperature to allow labeled oligonucleotides to anneal to complementary regions. In certain embodiments, melting comprises using a buffer or solution to melt the clusters, followed by using a hybridization buffer to allow complementary oligonucleotides to anneal to the clusters.

Many embodiments measure dissociation curves on the sequencing chip at 162. Dissociation curves relate to any process to reduce structure within a query region, such as melting (e.g., by increasing the temperature on the sequencing chip or flow cell). In some embodiments, a melt curve is generated by slowly increasing temperature to the flow cell or chip while imaging the chip to identify changes in fluorescence of molecules. In some embodiments, the temperature is adjusted continuously over a time course during imaging, while other embodiments increase temperature by a set amount of degrees and allowing the nucleic acids on the flow cell or sequencing chip to equilibrate to the new temperature prior to imaging. In some embodiments, the flow cell or sequencing chip initially starts at a temperature of 4° C., 5° C., 7° C., 10° C., 12° C., 15° C., 17° C., or 20° C. In certain embodiments the final temperature is at least 60° C., 65° C., 70° C., 75° C., 80° C., 85° C., 90° C., 95° C., or 100° C. In certain embodiments, the temperature is increased in increments of 1° C., 1.5° C., 2° C., 2.5° C., 3° C., 3.5° C., 4° C., 4.5° C., or 5° C. Temperature can be increased on a sequencing chip or flow cell via many means, including increasing a temperature of a buffer being perfused through a sequencing chip or flow cell and/or by using a heating plate in physical contact with the sequencing chip or flow cell.

Other embodiments generate dissociation curves by altering pH or altering composition and/or concentration of salt, buffer, protein, and/or organic molecules, where composition refers to the presence or absence of specific molecules within the solution. Altering pH can be accomplished by using various acids and/or bases, such as hydrochloric acid, acetic acid, sodium hydroxide, and/or other commonly used acids and bases. Additionally, exemplary salts include sodium chloride (NaCl), potassium chloride (KCl), and/or any other salt common to physiological or experimental environments. Exemplary buffers include sodium phosphate, sodium bisphosphate, sodium carbonate, sodium bicarbonate, potassium phosphate, potassium bisphosphate, potassium carbonate, potassium bicarbonate, sodium acetate, potassium acetate, and/or any other buffer commonly used. Furthermore, exemplary organic molecules include formamide, dimethyl sulfoxide, and/or any other organic molecule commonly used in reaction conditions. Additional embodiments include organic molecules to be analyzed, which may also have an effect on thermodynamics and/or conformation, including androgen and androgen-like molecules. Altering pH, salt, buffer, and/or organic molecule composition and/or concentration can be accomplished by altering such parameter of a solution being perfused through a sequencing chip or flow cell.

It should be noted that various embodiments may alter more than one parameter selected from temperature, pH, salt, buffer, and/or organic molecules. For example, buffer and salt can be altered simultaneously; temperature and pH can be altered simultaneously; temperature, pH, salt, buffer, and organic molecules can be altered simultaneously; and/or any other combination of noted parameters can be altered simultaneously.

In many embodiments, dissociation curves are measured quantitatively based on the change in fluorescence of a particular cluster over the melting process. For example, changes in fluorescence color (e.g., from FRET) may indicate melting of a structure in the nucleic acid (e.g., denaturation of a hairpin), which would increase the distance between two fluorophores. Additionally, an increase in fluorescence can indicate melting, where one labeled oligonucleotide comprises a quencher, thus as the distance between the fluorophore and the quencher, fluorescence will increase.

### Assessment of Nucleic Acid Interactions

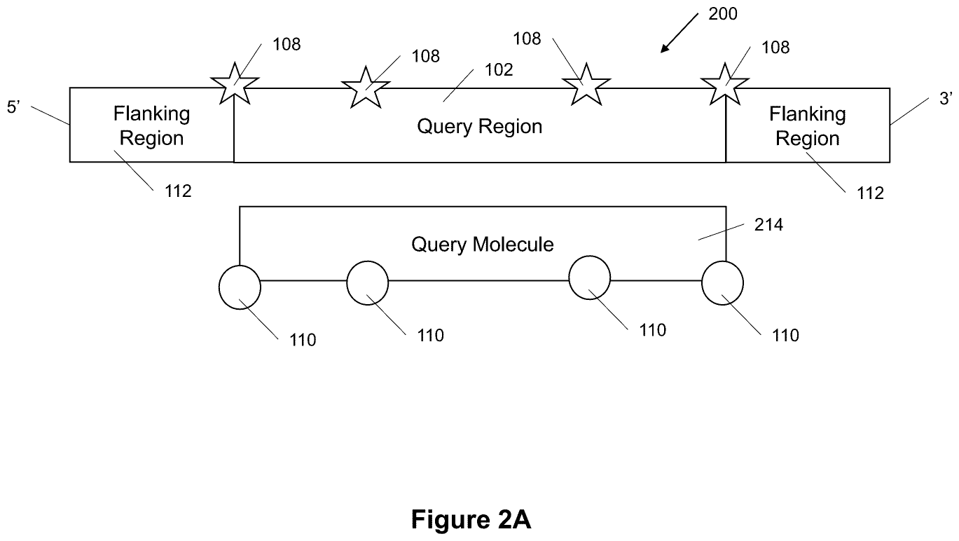

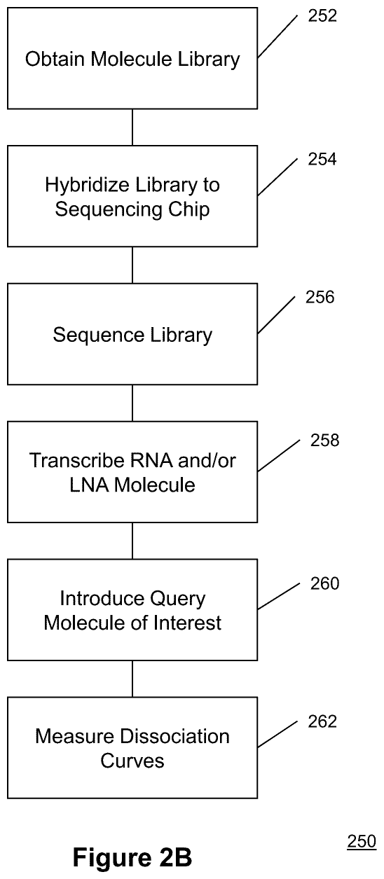

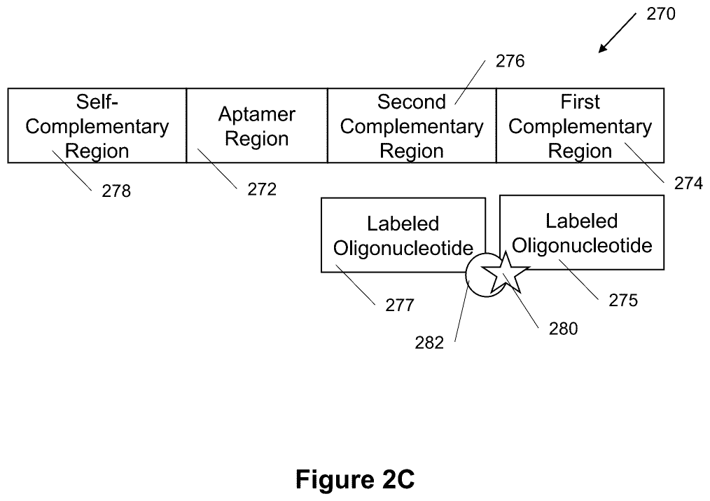

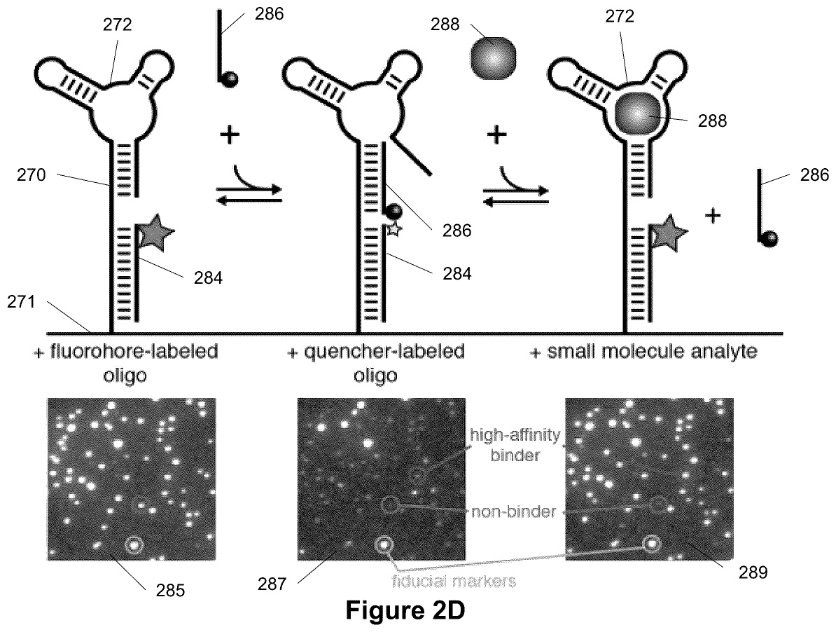

Additional embodiments are capable of identifying thermodynamics and conformation of nucleic acid interactions. Such interactions can be between nucleic acid molecules of either the same type or different types (e.g., RNA-RNA, RNA-DNA, RNA-LNA, DNA-DNA, DNA-LNA, LNA-LNA, and/or any other form of nucleic acid). Further embodiments assess interactions between nucleic acid molecules and other molecules, including (but not limited to) other nucleic acids, proteins, peptides, carbohydrates, organic compounds (including medicinal compounds, “small” molecules, drugs, etc.). Some embodiments assess interactions between aptamers and analytes (or any other molecules capable of interacting with or binding to an aptamer). FIG. 2A illustrates an exemplary nucleic acid molecule 200 for assessing interactions between multiple molecules, in accordance with many embodiments. Similar to molecule 100 described in FIG. 1A, FIG. 2A illustrates a nucleic acid molecule 200 possessing a 5′-end, a 3′-end, a query region 102, and one or more flanking regions 112, each possessing similar properties as described in regard to FIG. 1A. In many embodiments, the nucleic acid molecule 200 is single stranded or double stranded. In various embodiments, nucleic acid molecule 200 is selected from DNA, RNA, LNA, and/or a combination thereof. It will be understood to one of skill in the art that in a double stranded nucleic acid molecule, only one strand is considered in design of a molecule 200. Thus, while a double stranded molecule will include a complementary strand with its 3′-end paired a 5′-end, such a strand is understood as part of a molecule 200.

Various embodiments include a label 108 located on query region 102. Certain embodiments can be located at one end (e.g., 5′-end or 3′-end) of query region 102. Some embodiments include more than one label 108, such that a label 108 can exist at any combination of the 5′-end, the 3′-end, and/or one or more locations within the query region 102.

Additional embodiments can include a query molecule 214 that may interact with query region 102. A query molecule 214 can be another nucleic acid molecule, a protein, a peptide, a carbohydrate, an organic compound, any other type of molecule, and/or combinations thereof. In many embodiments, the query molecule is labeled with a tag 110. Tag 110 can be placed at a particular location on query molecule 214, such as a terminal location (e.g., 5′-end, 3′-end, N-terminus, C-terminus, etc.), at an internal location, or at another location on query molecule 214. Various embodiments include multiple tags 110, where the tags can be located at any position previously noted (e.g., terminal, internal, etc.). Certain query molecules 214 may possess inherent properties, such as absorbance, excitation/emission, etc. In such embodiments, one or more tags 110 may not be necessary as the query molecule 214 itself can act to amplify, positively interfere, negatively interfere, suppress, and/or otherwise interact with tag 108.

Tags 108, 110 can have similar properties as described in regard to FIG. 1A, including (but not limited to) fluorescence and/or absorbance, such as fluorophores and quenchers.

Many embodiments can utilize molecules, such as nucleic acid molecule 200 in to screen for interactions. Turning to FIG. 2B, an exemplary method 250 for high throughput interaction screening is illustrated. At 252, many embodiments obtain a library of molecules, where each molecule in the library comprises a query sequence of interest. A library in various embodiments includes any number of molecules, such as 1 molecule, 2 molecules, 3 molecules, 5 molecules, 10 molecules, 25 molecules, 50 molecules, 75 molecules, 100 molecules, 150 molecules, 200 molecules, 250 molecules, 300 molecules, 350 molecules, 400 molecules, 450 molecules, 500 molecules, 750 molecules, 800 molecules, 850 molecules, 900 molecules, 950 molecules, 800 molecules, 900 molecules, 1000 molecules, 1250 molecules, 1500 molecules, 1750 molecules, 2000 molecules, 2500 molecules, 5000 molecules, 10,000 molecules, 15,000 molecules, 20,000 molecules, 25,000 molecules, 50,000 molecules, 100,000 molecules, 250,000 molecules, 500,000 molecules, 1,000,000 molecules, 2,000,000 molecules, 5,000,000 molecules, 10,000,000 molecules, or more molecules. In many embodiments, the query sequence of interest is designed to interact with another molecule, such as described herein and/or known in the art. Certain embodiments possess a structure similar to the exemplary molecule illustrated in FIG. 2A (e.g., nucleic acid molecule 200). In many embodiments, the molecule is a nucleic acid molecule, such as a DNA molecule, an RNA molecule, and LNA molecule, and/or a combination of multiple types of nucleic acid molecules. In many embodiments, the length varies between 10 and 300 nucleotides in length, such as as 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, 100 nucleotides, 110 nucleotides, 120 nucleotides, 125 nucleotides, 130 nucleotides, 140 nucleotides, 150 nucleotides, 160 nucleotides, 170 nucleotides, 175 nucleotides, 180 nucleotides, 190 nucleotides, 200 nucleotides, 225 nucleotides, 250 nucleotides, 275 nucleotides, or 300 nucleotides. The ability of a query sequence of interest and another molecule of interest can be assessed.

Many embodiments obtain the library as DNA molecule analogs of RNA and/or LNA sequences, while certain embodiments obtain the library as RNA molecules and/or LNA molecules. Further embodiments synthesize the molecules directly as DNA, RNA and/or LNA via any applicable means, such as transcription, polymerization, and/or ordering such sequences from third party sources or vendors. Various embodiments amplify the molecular library to increase the copy number of sequences, through means, such as PCR or other known methodologies.

At 254, various embodiments hybridize the molecular library to a sequencing chip or flow cell. Various sequencing platforms operate differently, such that certain molecules possess a sequencing chip (e.g., Ion Torrent, Roche 454), while others possess a flow cell (e.g., Illumina). Some embodiments utilize adapter sequences within the molecules to allow the molecules to hybridize to the sequencing chip or flow cell. Many embodiments utilize an Illumina flow cell, such as a flow cell from a Genome Analyzer, MiSeq, HiSeq, HiScan, iSeq, MiniSeq, NextSeq, NovaSeq and/or any other Illumina sequencing platform. Once hybridized to an Illumina flow cell, certain embodiments generate clusters on the Illumina flow cell. Such embodiments follow known methods of amplifying molecules on a flow cell. On other platforms, similar processes can be undertaken to generate molecules attached to a sequencing chip-such processes are specific to the sequencing platform and can be identified in manuals or other literature specific to such platforms.

Certain embodiments sequence the molecules at 256. Such methods are known in the art. In the situation of many sequencing platforms, including Illumina platforms, sequencing reveals the location and/or coordinates of specific sequences, which correlate to individual molecules within the library. Such sequences are identified by the sequencing process.

Embodiments measuring interactions between RNA and/or LNA and an additional molecule thermodynamics transcribe an RNA molecule anchored to the flow cell or sequencing chip at 258. Such embodiments can generate the RNA via methods, such as those described in She, R., Chakravarty, A. K., Layton, C. J., Chircus, L. M., Andreasson, J. O., Damaraju, N., McMahon, P. L., Buenrostro, J. D., Jarosz, D. F. and Greenleaf, W. J. (2017) Comprehensive and quantitative mapping of RNA-protein interactions across a transcribed eukaryotic genome. Proc Natl Acad Sci USA, 114, 3619-3624; the disclosure of which is hereby incorporated by reference in its entirety. LNA or other forms of nucleic acid molecules can be generated with similarly applicable methods. However, measuring interactions between DNA and another molecule, such methods may not be necessary, if the molecules are already in DNA form.

At 260, many embodiments introduce one or more molecules of interest (e.g., query molecule 214, FIG. 2A) to the molecules. In various embodiments, when a molecule of interest interacts with a query sequence, tags (e.g., tags 108, 110, FIG. 2A) located on the query sequence of interest and the query molecule of interest the tags are within a specified distance of each other such that the tags can interact (e.g., via FRET, quenching, and/or any other form of interaction described herein).

Further embodiments measure dissociation curves on the sequencing chip at 262. Dissociation curves relate to any process to reduce the interaction between a query region and a query molecule, such as melting (e.g., by increasing the temperature on the sequencing chip or flow cell). In some embodiments, a melt curve is generated by slowly increasing temperature to the flow cell or sequencing chip while imaging the chip to identify changes in fluorescence of molecules. In some embodiments, the temperature is adjusted continuously over a time course during imaging, while other embodiments increase temperature by a set amount of degrees and allowing the nucleic acids on the flow cell or sequencing chip to equilibrate to the new temperature prior to imaging. In some embodiments, the flow cell or sequencing chip initially starts at a temperature of 4° C., 5° C., 7° C., 10° C., 12° C., 15° C., 17° C., or 20° C. In certain embodiments the final temperature is at least 60° C., 65° C., 70° C., 75° C., 80° C., 85° C., 90° C., 95° C., or 100° C. In certain embodiments, the temperature is increased in increments of 1° C., 1.5° C., 2° C., 2.5° C., 3° C., 3.5° C., 4° C., 4.5° C., or 5° C. Temperature can be increased on a sequencing chip or flow cell via many means, including increasing a temperature of a buffer being perfused through a sequencing chip or flow cell and/or by using a heating plate in physical contact with the sequencing chip or flow cell.

Other embodiments generate dissociation curves by altering pH or altering composition and/or concentration of salt, buffer, and/or organic molecules, where composition refers to the presence or absence of specific molecules within the solution. Altering pH can be accomplished by using various acids and/or bases, such as hydrochloric acid, acetic acid, sodium hydroxide, and/or other commonly used acids and bases. Additionally, exemplary salts include sodium chloride (NaCl), potassium chloride (KCl), and/or any other salt common to physiological or experimental environments. Exemplary buffers include sodium phosphate, sodium bisphosphate, sodium carbonate, sodium bicarbonate, potassium phosphate, potassium bisphosphate, potassium carbonate, potassium bicarbonate, sodium acetate, potassium acetate, and/or any other buffer commonly used. Furthermore, exemplary organic molecules include formamide, dimethyl sulfoxide, and/or any other organic molecule commonly used in reaction conditions. Altering pH, salt, buffer, organic molecule, and/or small molecules (e.g., drugs, medicinal compounds, or organic and/or inorganic compounds) composition and/or concentration can be accomplished by altering such parameter of a solution being perfused through a sequencing chip or flow cell.

It should be noted that various embodiments may alter more than one parameter selected from temperature, pH, salt, buffer, and/or organic molecules. For example, buffer and salt can be altered simultaneously; temperature and pH can be altered simultaneously; temperature, pH, salt, buffer, and organic molecules can be altered simultaneously; and/or any other combination of noted parameters can be altered simultaneously.

In many embodiments, dissociation curves are measured quantitatively based on the change in fluorescence of a particular cluster over the melting process. For example, changes in fluorescence color (e.g., from FRET) may indicate dissociate of a query sequence and query molecule, which would increase the distance between two fluorophores. Additionally, an increase in fluorescence can indicate dissociation, where one of the query sequences and the query molecule comprises a quencher, thus as the distance between the fluorophore and the quencher, fluorescence will increase.

### Calibrating Data for Temperature Effects

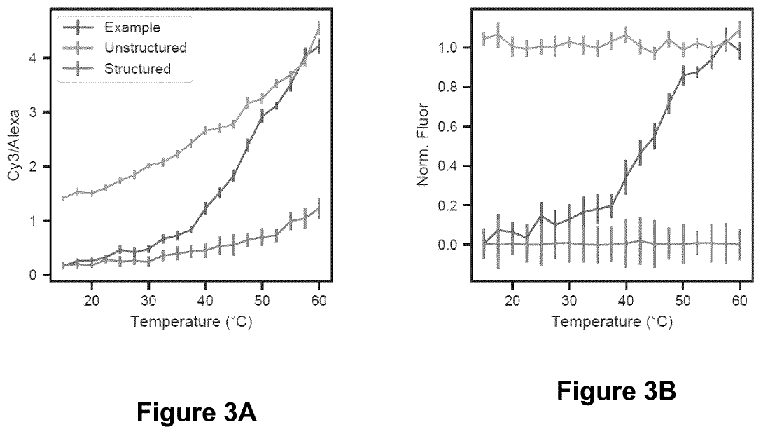

Various embodiments further calibrate fluorescence based on the temperatures of the flow cell or sequencing chip. For example, some fluorophores may have a thermal effect (e.g., increased or decreased fluorescence at different temperatures. Such effects can be controlled for based on control molecules (e.g., molecules possessing a complementary region for only one labeled oligonucleotide). For example, FIG. 3A illustrates exemplary fluorescence of an unstructured control molecule, a structured control molecule (e.g., a molecule that should not melt), and an example of a meltable structure. The changes in fluorescence of the structured and unstructured control molecules in FIG. 3A indicate the thermal effect of some fluorophores, which can affect accuracy of a melt cover (e.g., the example melt curve). FIG. 3B illustrates exemplary normalization of fluorescence. Based on such calibration, embodiments can identify a fraction of unfolded molecules at a particular temperature according to the following equation:

$$Fraction Unfolded = \frac{F - {F}_{min}}{{F}_{max} - {F}_{min}}$$

Where Fₘᵢₙ is the minimum fluorescence for a set of control constructs designed to remain folded at increasing temperatures. Fₘₐₓ is the maximum fluorescence obtained, determined as the average over a set of unstructured controls included.

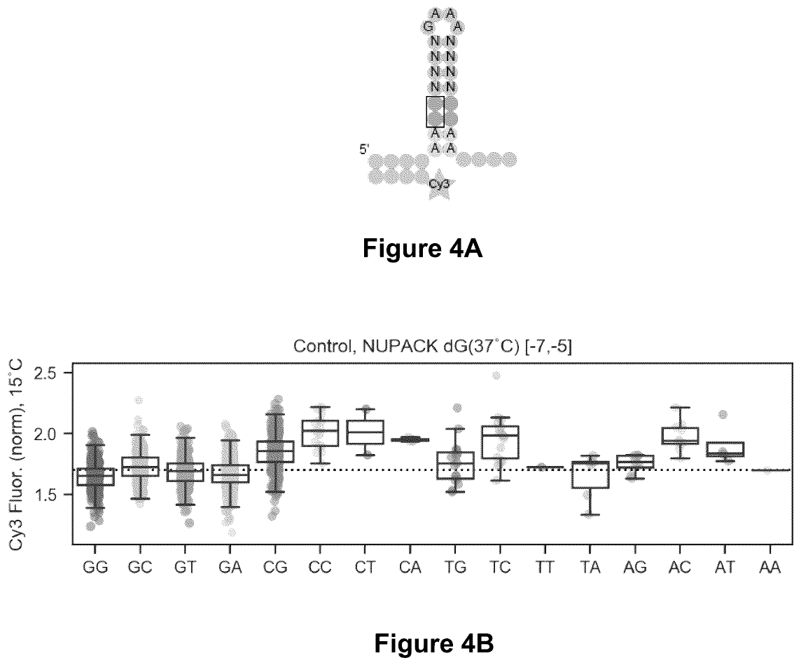

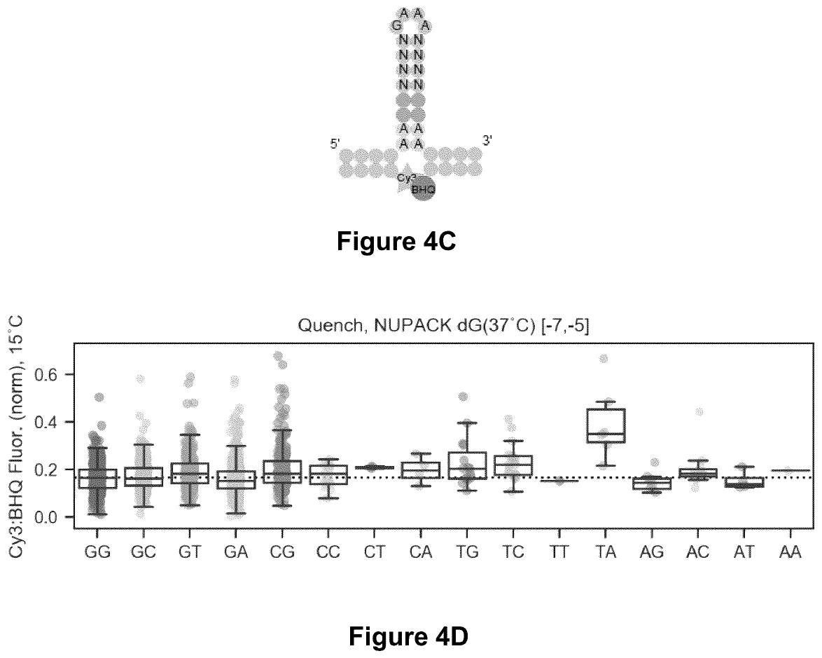

Additionally, certain embodiments control for effects caused by nucleotides in the vicinity of a fluorophore. FIGS. 4A-4D illustrates exemplary data of normalized fluorescence of the fluorophore Cy3 in the vicinity of various nucleotides. Specifically, FIG. 4A illustrates a control molecule possessing only an upstream complementary region and complementary, fluorescently labeled oligonucleotide, and FIG. 4B illustrates normalized fluorescence of Cy3 for various nucleotides in the boxed region of FIG. 4A. Similarly, FIG. 4C illustrates a molecule possessing an upstream complementary region with a complementary, fluorescently labeled oligonucleotide and a downstream complementary region with a complementary, quencher-labeled oligonucleotide. FIG. 4D normalized fluorescence of Cy3 for various nucleotides in the boxed region of FIG. 4D, indicating that the nucleotide effect is limited in the presence of the quencher.

### Training a Machine Learning Model

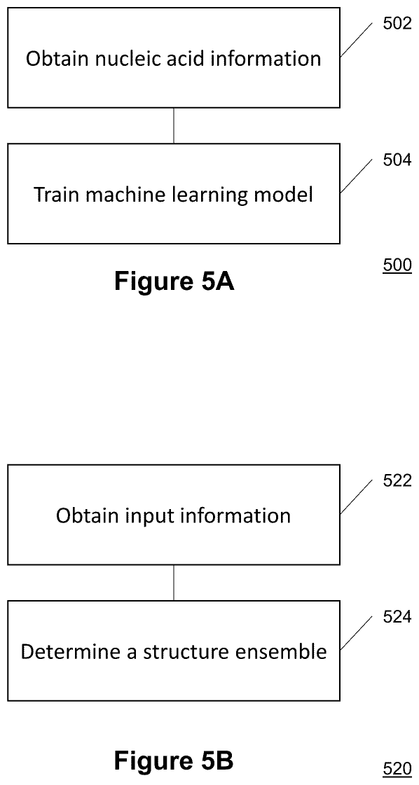

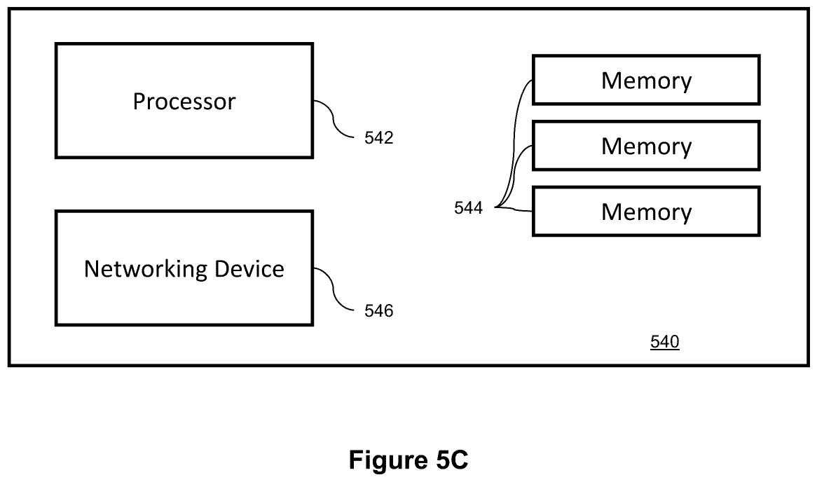

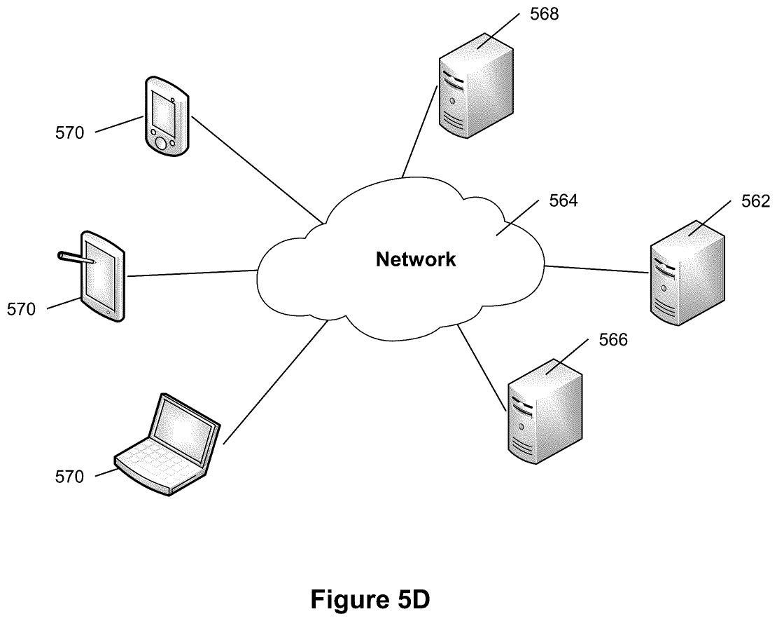

Once thermodynamic measurements are obtained from many different sequences, various embodiments are able to predict thermodynamic properties of nucleic acid molecules and/or interaction properties of nucleic acid molecules with other molecules. For example, the high throughput screening data can be used as training data for a machine learning model or other system to predict thermodynamic stability or other property from a nucleic acid sequence. Turning to FIG. 5A, an exemplary method 500 for training a machine learning model, such that the machine learning model can determine nucleic acid thermodynamics, is illustrated in accordance with various embodiments.

Many embodiments obtain nucleic acid information at 502. In such embodiments, the nucleic acid information includes a nucleic acid sequence, such as DNA, RNA, and/or LNA. Various embodiments obtain sequence and thermodynamic information for multiple nucleic acid sequences, while certain embodiments obtain sequence information and interaction data. The multiple nucleic acid sequences can be a library of nucleic acid sequences, where the library includes 1 sequence, 2 sequences, 3 sequences, 5 sequences, 10 sequences, 25 sequences, 50 sequences, 75 sequences, 100 sequences, 150 sequences, 200 sequences, 250 sequences, 300 sequences, 350 sequences, 400 sequences, 450 sequences, 500 sequences, 750 sequences, 800 sequences, 850 sequences, 900 sequences, 950 sequences, 800 sequences, 900 sequences, 1000 sequences, 1250 sequences, 1500 sequences, 1750 sequences, 2000 sequences, 2500 sequences, 5000 sequences, 10,000 sequences, 15,000 sequences, 20,000 sequences, 25,000 sequences, 50,000 sequences, 100,000 sequences, 250,000 sequences, 500,000 sequences, 1,000,000 sequences, 2,000,000 sequences, 5,000,000 sequences, 10,000,000 sequences, or more sequences.

In certain embodiments, the thermodynamic data and/or interaction data is generated experimentally. In some embodiments, the experimental data is experimentally determined via high throughput means, such as those described herein (e.g., method 150, FIG. 1D or method 250, FIG. 2B.)

As the environmental conditions can affect the dissociation and thermodynamics, certain embodiments further include environmental parameters, for which the experimental data was generated, such as one or more of temperature, pH, buffers, salts, and/or organic molecule composition and/or concentration, and/or any other component or parameter within the experimental measurement. Some embodiments can include multiple experimental data for each nucleic acid molecule—for example, for one nucleic acid sequence, multiple experimentally determined dissociations are included in the nucleic acid information.

At 504, further embodiments train a machine learning model to determine thermodynamics and/or interactions using the nucleic acid information. The machine learning model can be selected from any appropriate model type and trained via any relevant learning technique, as appropriate, such as a sparse technique or a non-sparse technique and a regression technique or a classification technique. Accordingly, the learning technique is preferably chosen from among the group consisting of: a sparse regression technique, a sparse classification technique, a non-sparse regression technique and a non-sparse classification technique.

As an example, the learning technique is therefore chosen from among the group consisting of: a linear or logistic linear regression technique with L1 or L2 regularization, such as the Lasso technique or the Elastic Net technique; (see e.g., Tibshirani and Zou and Hastie; cited above;) a model adapting linear or logistic linear regression techniques with L1 or L2 regularization, such as the Bolasso technique (see e.g., Bach, Francis R. “Bolasso: model consistent lasso estimation through the bootstrap.” Proceedings of the 25th international conference on Machine learning. 2008; the disclosure of which is hereby incorporated by reference herein in its entirety), the relaxed Lasso (see e.g., Meinshausen, Nicolai. “Relaxed lasso.” Computational Statistics & Data Analysis 52.1 (2007): 374-393; the disclosure of which is hereby incorporated by reference herein in its entirety;) the random-Lasso technique (see e.g., Wang, Sijian, et al. “Random lasso.” The annals of applied statistics 5.1 (2011): 468; the disclosure of which is hereby incorporated by reference herein in its entirety;) the grouped-Lasso technique (see e.g., Friedman, Jerome, Trevor Hastie, and Robert Tibshirani. Applications of the lasso and grouped lasso to the estimation of sparse graphical models. Technical report, Stanford University, 2010; the disclosure of which is hereby incorporated by reference herein in its entirety;) the LARS technique (see e.g., Eyraud, Remi, Colin De La Higuera, and Jean-Christophe Janodet. “LARS: A learning algorithm for rewriting systems.” Machine Learning 66.1 (2007): 7-31; the disclosure of which is hereby incorporated by reference herein in its entirety;) a linear or logistic linear regression technique without L1 or L2 regularization; a non-linear regression or classification technique with L1 or L2 regularization; a Decision Tree technique; a Random Forest technique; a Support Vector Machine technique, also called SVM technique; a Neural Network technique (including graph Neural Network); and a Kernel Smoothing technique.

### Small Molecule Sensing

Certain embodiments can be utilized to detect small molecules in a massively parallel fashion. Such embodiments can be used to determine composition and/or concentration of a complex mixture. Such small molecules can come from environmental samples (e.g., soil, air, water, etc.), biological samples (e.g., saliva, blood, urine, fecal, tissue, etc.) and/or any other sample that includes small molecules. Such small molecules can be metabolites, medicinal compounds, organic compounds, and/or any other molecule of interest. Such embodiments can be referred to as “molecular noses” for the ability to identify numerous molecules simultaneously, either qualitatively and/or quantitatively. Such embodiments can combine aspects of molecules 100 and 200. FIG. 2C illustrates an exemplary schematic of a molecular nose in accordance with many embodiments. Molecule 270 of many embodiments is a nucleic acid molecule, such as DNA, RNA, and/or LNA.

In many embodiments, molecule 270 includes an aptamer region 272. An aptamer region in this context is a nucleic acid molecule that is capable of binding and/or interacting with a particular molecule-such interactions can be reversible and/or of various strengths or affinities. Some interactions can create a conformational change in an aptamer, including in aptamer region 272.

Additional embodiments include complementary regions 274, 276 that are capable of forming a complement with labeled oligonucleotides 275, 277 and/or self-complementary region 278. For example, first complementary region 274 may pair with a labeled oligonucleotide 275, while second complementary region may pair with labeled oligonucleotide 277. Additionally, self-complementary region 278 may pair with one or both complementary regions 274, 276. In numerous embodiments, self-complementary region 278 and labeled oligonucleotide 277 both bind to second complementary region 276.

In many embodiments, labeled oligonucleotides 275, 277 include a tag 280, 282. As noted herein, labels 280, 282 can be fluorophores and/or quenchers. In some embodiments, tag 280 is a fluorophore, while tag 282 is a quencher. In such embodiments, label 280 can be allowed to fluoresce when labeled oligonucleotide 277 is not present or when self-complementary region 278 is paired with second complementary region 276.

Turning to FIG. 2D, an exemplary mechanism for a molecular nose is illustrated. In particular, FIG. 2D illustrates molecule 270 attached to a sequencing chip or flow cell 271. Sequencing can identify the location or coordinates of each molecule 270, including its respective aptamer region 272, on a sequencing chip or flow cell 271. Aptamer region 272 can fold to its appropriate conformation to be functional as an aptamer. Additionally, locations of various molecules can be verified when a fluorophore-labeled oligonucleotide 284, such as illustrated in image 285. A quencher-labeled oligonucleotide 286 can be hybridized to the molecule 270, which will suppress the fluorescence of a fluorophore-labeled oligonucleotide 284, such as illustrated in image 287. Finally, when a sample is introduced to a sequencing chip or flow cell 271, a complementary molecule 288 can bind to an aptamer region 272, which changes the conformation of the aptamer region, which can dislodge quencher-labeled oligonucleotide 286, reallowing fluorescence, such as illustrated in image 289.

It should be noted that some embodiments may include fiducial markers or molecules 270 that stay fluorescent even when a quencher-labeled oligonucleotide 286 is introduced. Such markers can be used to align or confirm positions of images during a sensing process. Additionally, some molecules 270 may be “non-binders,” such as when a molecule 270 does not have a complementary molecule 288 that would cause displacement of a quencher-labeled oligonucleotide 286, which leads to no fluorescence. Finally, certain molecules may be a high-affinity binder, such that fluorescence is much brighter than other molecules 270.

Determining Nucleic Acid Thermodynamics and/or Interactions

Many embodiments can be used to determine nucleic acid thermodynamics and/or interactions using a machine learning model. FIG. 5B illustrates an exemplary method 520 to determine nucleic acid thermodynamics for one or more sequences in accordance with many embodiments. Such embodiments can accept one or more nucleic acid sequences (e.g., DNA, RNA, and/or LNA) to determine their thermodynamics, such as described herein.

Many embodiments obtain input information at 522. The input information of such embodiments can include one or more nucleic acid sequences and/or query molecules, as appropriate for the intended purpose. As noted herein, the one or more nucleic acid sequences can be DNA, RNA, LNA, and/or a combination of DNA, RNA, and LNA sequences. Query molecules can include other nucleic acids, proteins, peptides, carbohydrates, organic compounds (including medicinal compounds, “small” molecules, drugs, etc.). Further embodiments allow for additional inputs such as one or more environmental condition, including one or more of temperature, pH, buffers, salts, and/or organic molecule composition and/or concentration, and/or any other component or parameter of interest. Certain embodiments allow for multiple environmental conditions to be set in an alternative form—e.g., 20° C. and 37° C.—such that multiple determinations can be made automatically. The multiple conditions input can be extended to a set of specific conditions or a range of conditions (e.g., from 20° C. to 37° C.), such that thermodynamics can be determined as it changes under varying conditions.

At 524, additional embodiments determine thermodynamics for the one or more input sequences. In many embodiments, the determination utilizes a machine learning model such as described herein. Many of these machine learning models can be trained such as described herein.

### Computer Executed Embodiments

Processes that provide the systems and methods to determine nucleic acid thermodynamics in accordance with some embodiments are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes in accordance with some embodiments are shown in FIG. 5C. One skilled in the art will recognize that computing devices or systems may include other components that are omitted for brevity without departing from described embodiments. A computing device 540 in accordance with such embodiments comprises a processor 542 and at least one memory 544. Memory 544 can be a non-volatile memory and/or a volatile memory, and the processor 542 is a processor, microprocessor, controller, or a combination of processors, microprocessor, and/or controllers that performs instructions stored in memory 544. Such instructions stored in the memory 544, when executed by the processor, can direct the processor, to perform one or more features, functions, methods, and/or steps as described herein. Any input information or data can be stored in the memory 544—either the same memory or another memory. In accordance with various other embodiments, the computing device 540 may have hardware and/or firmware that can include the instructions and/or perform these processes.

Certain embodiments can include a networking device 546 to allow communication (wired, wireless, etc.) to another device, such as through a network, near-field communication, Bluetooth, infrared, radio frequency, and/or any other suitable communication system. Such systems can be beneficial for receiving data, information, or input from another computing device and/or for transmitting data, information, or output to another device.

Turning to FIG. 5C, an embodiment with distributed computing devices is illustrated. Such embodiments may be useful where computing power is not possible at a local level, and a central computing device (e.g., server) performs one or more features, functions, methods, and/or steps described herein. In such embodiments, a computing device 562 (e.g., server) is connected to a network 564 (wired and/or wireless), where it can receive inputs from one or more computing devices, including clinical data from a records database or repository 566, data provided from a laboratory computing device 568, and/or any other relevant information from one or more other remote devices 570. Once computing device 562 performs one or more features, functions, methods, and/or steps described herein, any outputs can be transmitted to one or more computing devices 566, 5668, 570 for entering into records or taking other action. Such actions can be transmitted directly to a medical professional (e.g., via messaging, such as email, SMS, voice/vocal alert) for such action and/or entered into medical records.

In accordance with still other embodiments, the instructions for the processes can be stored in any of a variety of non-transitory computer readable media appropriate to a specific application.

### Exemplary Embodiments

Although the following embodiments provide details on certain embodiments of the inventions, it should be understood that these are only exemplary in nature, and are not intended to limit the scope of the invention.

### Example 1: Characterizing Nucleic Acid Thermodynamics Through High-Throughput Measurements

Background: Base-pairing in DNA and RNA molecules underlies many critical processes in biology, including signaling, viral replication and packaging, catalysis, structure of noncoding RNAs, as well as in biotechnology, such as design of improved constructs and protocols for PCR amplification. Numerous algorithms have been developed to predict DNA and RNA secondary structure thermodynamics, many of which make use of parameters inferred from optical melting experiments on a handful of constructs. Recent work with more high throughput readouts of nucleic acid structure have demonstrated that algorithms based on these optical melting experiments perform poorly at predicting experimental observables such as RNA-protein binding constants and RNA structure mapping experiments. A major bottleneck limiting prior model development is the throughput available to methods that characterize DNA and RNA duplexes one-by-one.

Methods: Library assembly and sequencing. Designed library variants were synthesized into DNA by Twist Biosciences (South San Francisco, CA). The synthesized oligo pool was amplified using internal primers to enrich for full-length library variants. The PCR reaction consisted of: 1/100× dilution of the synthesized oligo pool (final concentration 0.01 nM), 200 nM of each primer (T7A1 library, D-TruSeqR2 Table S1), 1× Phire Hot Start II PCR Master Mix (ThermoFisher Scientific F125L). The reaction proceeded for 9 cycles of 98° C. for 10 seconds, 56° C. for 30 seconds, and 72° C. for 30 seconds. Reaction mixtures were purified using QIAquick PCR Purification Kit (Qiagen 28104) to remove primers and proteins, and eluted into 20 uL dilution buffer.

After initial amplification, the library was amplified with primers to bring in sequences compatible with Illumina sequencing. This five-piece assembly PCR included two outside primers and two adapter sequences. The PCR reaction consisted of 1 μl of the previous reaction, 137 nM of outside primers (short_C and short_D; Table S1), 3.84 nM of the adapter sequences (C-i7pr-bc-T7A1 and D_TruSeqR2; Table S1), 1× Phire Hot Start II PCR Master Mix (ThermoFisher Scientific F125L). The reaction proceeded for 14 cycles of 98° C. for 10 seconds, 56° C. for 30 seconds, and 72° C. for 30 seconds. Reactions were purified using the QIAquick PCR Purification Kit and quantified with a Qubit Fluorometer (ThermoFisher Scientific).

Imaging station setup. An imaging station was used to image the Miseq chip at increasing temperatures. This station was built from a combination of custom-designed parts from a disassembled Illumina genome analyzer IIx. Two channels were employed: the “red” channel used the 660 nm laser and 664 nm long pass filter (Semrock), and the “green” channel used the 50 nm laser and 590 nm band pass filter (Semrock). All images were taken with 600 ms exposure times at 150 mW fiber input laser power. Focusing at each temperature was achieved by sequentially adjusting the z-position and re-imaging the four corners of the flow cell; the adjusted z-positions were then fit to a plane.

Post-sequencing, the chip was washed with Cleavage buffer (100 mM Tris-HCl, 125 mM NaCl, 0.05% Tween20, 100 mM TCEP, pH 7.4) to remove residual fluorescence from the reversible terminators used i the sequencing reaction at 60° C. for 5 minutes. Any strands of DNA not covalently attached to the surface of the chip was removed by washing in 100% formamide at 55° C. The resulting single-stranded DNA fragments were incubated with 500 nM of the oligo Biotin_D_Read2 and FID in Hybridization buffer (5×SSC buffer (ThermoFisher 15557036), 5 mM EDTA, 0.05% Tween20) for 15 minutes at 60° C., subsequently the temperature was lowered to 40° C. for another 10 minutes. For RNA experiments, RNA was then generated using the protocol described in.

Measuring melt curves on chip. Following either ssDNA generation or RNA generation, the Cy-3 labeled fluor_oligo was annealed at 40° C. for 5 minutes, then the quench-labeled oligo quench_oligo, then the Alexa-labeled oligo red_oligo using the same protocol. The chip was imaged after each step to ensure that hybridization had occurred through increase or quenching of signal in the corresponding channel. The chip was then rinsed with Melt buffer (50 mM Na-Hepes pH 8.0, 25 mM NaCl). To quantify the melt curves, the image station temperature was then lowered to 15° C. For each temperature point, the system was allowed to equilibrate to the new temperature (5 minutes) before refocusing and imaging. The temperature was raised in 2.5° C. increments to a maximum temperature of 60° C.

Processing sequencing data. Sequencing data from Illumina Miseq was processed to extract tile and coordinates of each sequenced cluster. Forward and reverse paired-end reads were aligned using FLASH with default settings. Consensus sequences from FLASH were aligned to the Cy3 reverse complement sequence and Quench oligomer reverse complement sequence using a Needleman-Wunsch alignment (nwalign3). For consensus sequences that successfully aligned to both with p-value<1e-3, evaluated as described in, the variable region was extracted as the region between the two flanking regions and aligned to sequences in the reference library. A reference sequence was assigned based on the best-scoring alignment with a p-value<1e-6. The resulting clusters were used for fluorescence quantification in the downstream data analysis.

Fluorescence data processing and image fitting. Images taken during MANIfold experiments were mapped to sequencing data from the Illumina Miseq. First, sequencing data was processed to extract the tile and coordinates of each sequenced cluster. To match each sequence to its location on our imaging station, the sequencing data was cross-correlated to images in an iterative fashion to map coordinates to the images at sub-pixel resolution as in. Once the locations were determined, each cluster was fit to a 2D normal distribution to quantify its fluorescence.

Fluorescence normalization. Size normalization. To reduce inter-cluster variation in fluorescence measurements at each temperature point, the amount of unfolded constructs was normalized (measured in the green channel) by the total amount of ssDNA or RNA in that cluster (measured in the red channel). The red channel signal was clipped to the 1st and 99th percentile of the total distribution at that temperature, and any clusters that failed to quantify the red channel at this point were removed.

Construct normalization. To account for effects on Cy3 fluorescence due to resonance or quenching from nearby nucleotides in different constructs, each construct in the library also had a control version lacking a quench oligo. The signal from each quenched construct was then divided by the signal from its control construct.

Calculating unfolded fraction. We calculated the fraction unfolded at a given temperature f (unfolded, T) for each construct as:

$$f ( unfolded , T ) = \frac{F - {F}_{min}}{{F}_{max} - {F}_{min}}$$

Fₘᵢₙ is the minimum fluorescence for a set of control constructs designed to remain folded at increasing temperatures. Fₘₐₓ is the maximum fluorescence obtained, determined as the average over a set of unstructured controls included. Standard error for the above calculation was estimated via bootstrapping in the following way: For each bootstrapping replicate, each value in the equation above is resampled by sampling with replacement over the cluster members in the dataset and taking the median.

Fitting thermodynamic parameters via a two-state model. The data was first fit assuming a two-state model for melting. Under this model, the probability of the hairpin being unfolded can be written as:

$$p ( unfolded , T ) = \frac{1}{1 + exp ( - Δ G / {k}_{\text{?}} T )} = \frac{1}{1 + exp [ \frac{Δ \text{?}}{{k}_{B}} ( \frac{1}{{T}_{m}} - \frac{1}{T} ) ]} ,$$

$$\text{?} \text{indicates text missing or illegible when filed}$$

Where ΔG=ΔH (1−T/Tₘ) is the free energy of the folded state, ΔH is the enthalpy, and Tₘ is the melting temperature (point of inflection in the melt curve), and kg is Boltzmann's constant (0.00198 kcal/mol/K). For each construct, ΔH and Tₘ were fit using iterative nonlinear fitting (scipy). Error was calculated by bootstrapping over all the clusters per construct on the chip. Linear models in scikit-learn were used to test various nearest-neighbor models for predicting the resulting ΔG_{construct} values from ΔG_{features}.

Fitting thermodynamic parameters via an ensemble-aware model. To evaluate the goodness-of-fit for a two-state model, thermodynamic parameters were also fit by minimizing the loss function:

$${L}_{MAN \text{?} fold} = {[ ( 1 - f ( unfolded ) ) - p ( terminal base pairs formed ) ]}^{2} .$$

$$\text{?} \text{indicates text missing or illegible when filed}$$

Where f(unfolded, T) is the experimentally-determined fraction unfolded at a given temperature (described above). The quantity p(terminal base pairs formed, T) is evaluated as the ensemble-averaged probability of the last three base pairs of the construct forming. This training was implemented in the EternaFold codebase, and parameters fit using the LBFG-S method.

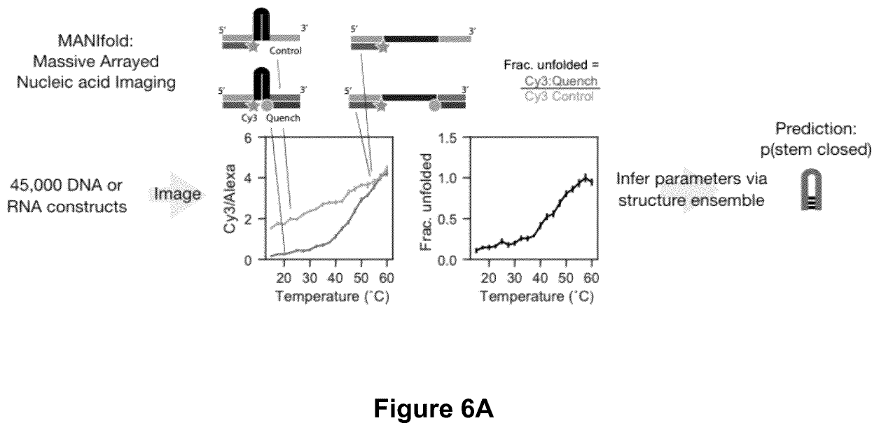

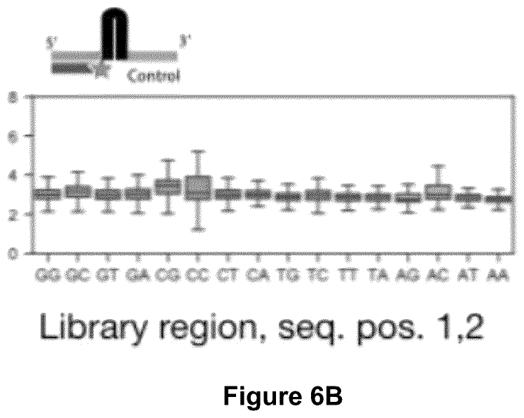

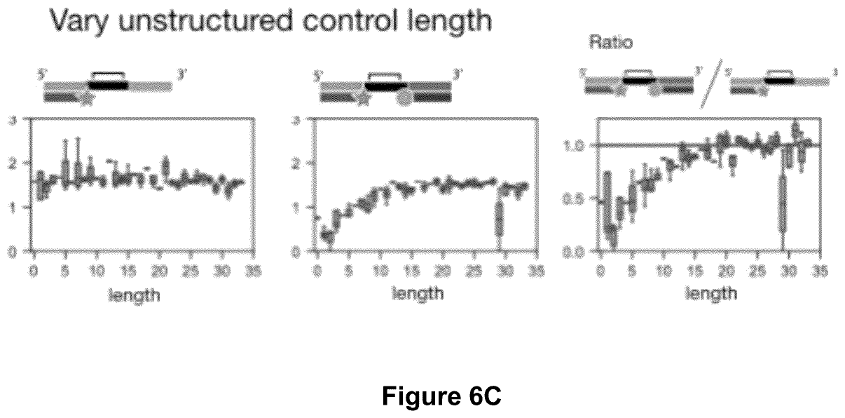

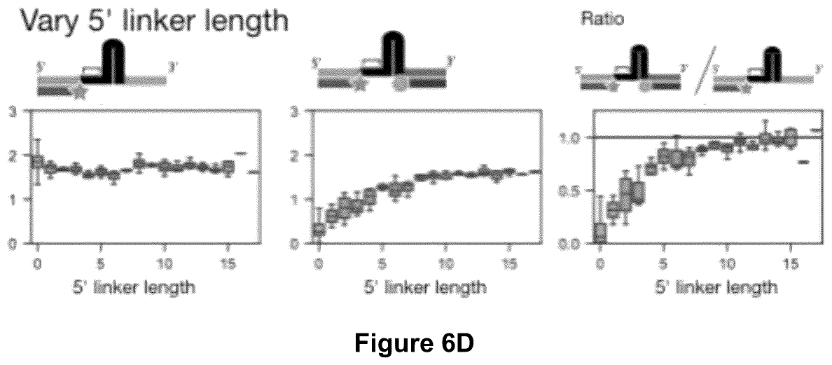

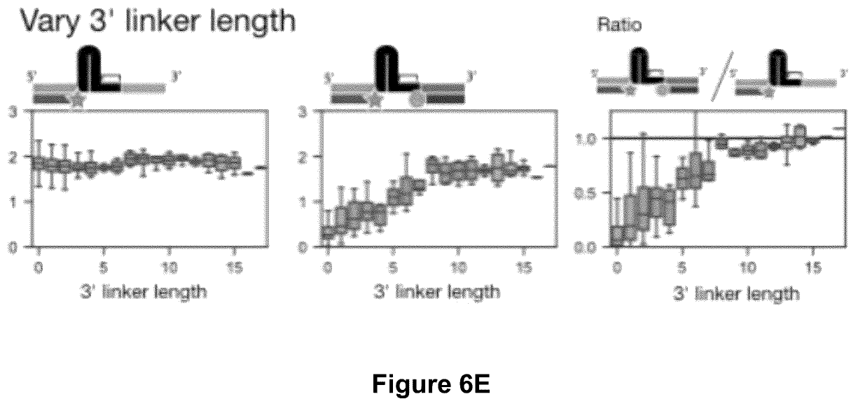

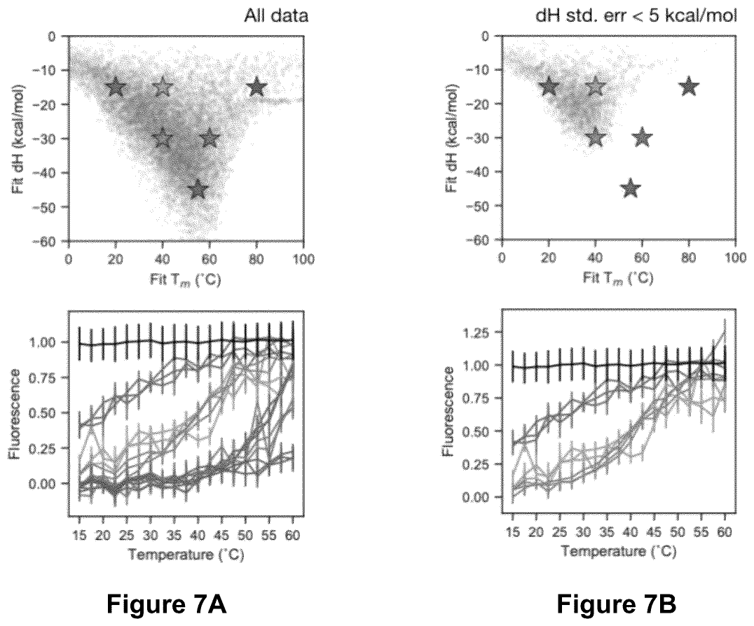

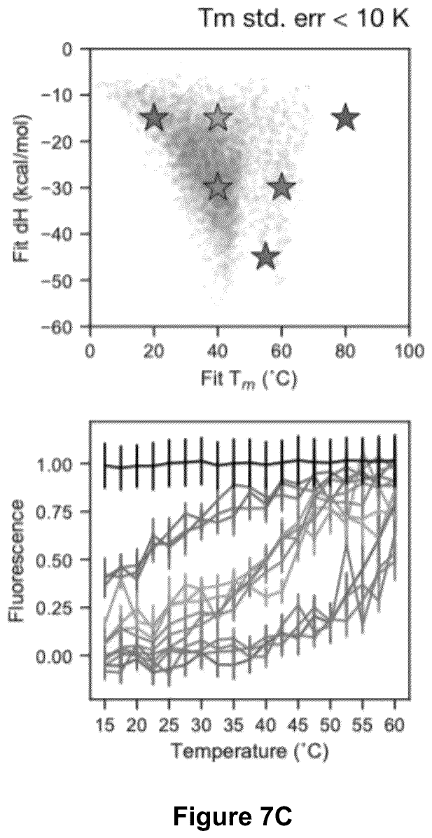

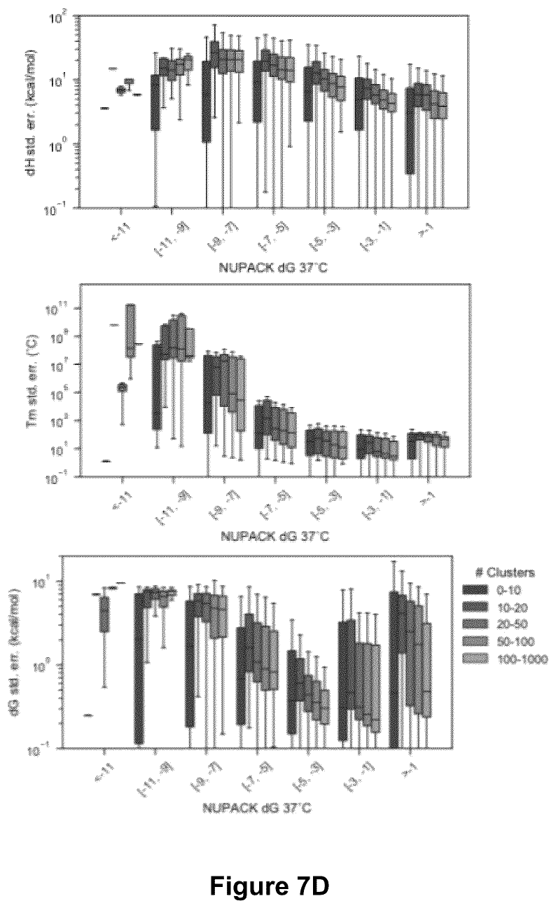

Results: MANIfold experimental design. The MANIfold library was designed to aim to quantitatively measure thermodynamics of nucleic acid hairpins unfolding in the following manner. Each construct characterized had two versions present in the library, a version with a fluorophore-annealing region upstream and a quench-annealing region downstream, the “quenched” version, and a version with a fluorophore-annealing region upstream and a region downstream orthogonal to the quench-functionalized oligomer, the “control” version (FIGS. 6A-6E). The control version of each construct was included to account for any sequence-specific amplification or quenching of the fluorophore by the variable library region. For both versions, the Cy3 fluorescence was normalized by the Alexa fluorescence to obtain a size-independent fluorescence (size-normalized fluorescence of a quenched and control construct in FIG. 6B). The size-normalized fluorescence of the quenched version was divided by the size-normalized fluorescence of the control version to obtain the quenching fraction for each construct (quenching fraction for example construct in FIG. 7C). An observed variation in the control fluorescence signal that indicated that a control would be needed to account for differing interactions between nucleotides and the Cy3 fluorophore (FIG. 7A).

It was desired to determine the persistence length of the system; i.e. at what nucleotide length does the quenching efficiency of an unstructured background match that of the control. A series of unstructured controls and constructs were designed with stems but varying the length of a polyA construct upstream or downstream of the stem. We observed that in all these three conditions, the quenching efficiency reached a value of 1 at roughly 16-17 nucleotides. This confirms that 1) the Cy3-BHQ pair is dominated by static quenching (rather than FRET, which would have further through-space quenching) and that 2) a partially-folded state shorter than ˜16 nucleotides would contribute to quenching.

Constructs designed to be unstructured (varying-length repeats of A, AG, AC, AAG, AAC, AAAG, AAAC) were included, and it was ascertained that this size- and sequence-normalized quenching fraction for unstructured constructs was constant across temperatures. The quenching fraction was then normalized to the minimum and maximum quenching fraction observed (see Methods). This resulted in an experimentally-determined frac. (unfolded) for each construct at each temperature.

After fluorophore and quench oligomers were annealed, the chip was imaged at temperature increases of 2.5° C. from 15 to 60° C. For initial analysis, the resulting frac. (unfolded) curves were fit to a two-state model to obtain dH and Tₘ values for each construct. FIG. 7A portrays the range of dH and Tₘ values fit. Subset of the data which had Tₘ error<10° C. and dH values <5 kcal/mol are portrayed in FIGS. 7B-6C respectively, indicating that the dynamic range of measurably dH and Tₘ is limited by the temperature range characterized. Standard error decreased with increasing number of variants, and was lowest for constructs in the center of the range of fit dGs (FIG. 7D).

For this initial library, only roughly 50% coverage of the constructs designed in the library were obtained. This did not allow for a comprehensive quantification of all the hypotheses designed in the library, but in the remainder of this chapter, we aim to address hypotheses about nucleic acid thermodynamics and fitting as best as possible. Subsequent analyses are based on constructs with more than 5 clusters in both the quenched and control construct, Tₘ standard error<10 K, dH standard error<5 K, and dG (37° C.) standard error<1 kcal/mol.

Testing the nearest-neighbor model for DNA Watson-Crick stacks. The nearest-neighbor model has been in wide use for nucleic acid thermodynamics since its introduction. It was aimed to quantitatively test the predictive power of the nearest-neighbor model vs. less complex (i.e., base-pairing only) and more-complex (i.e., triplet-stack features). To do this, constructs varying all Watson-crick pairs in the stem with constant regions to include all basepair triplets in a number of contexts were included, varying the lengths of stems included as well as closing base pairs.

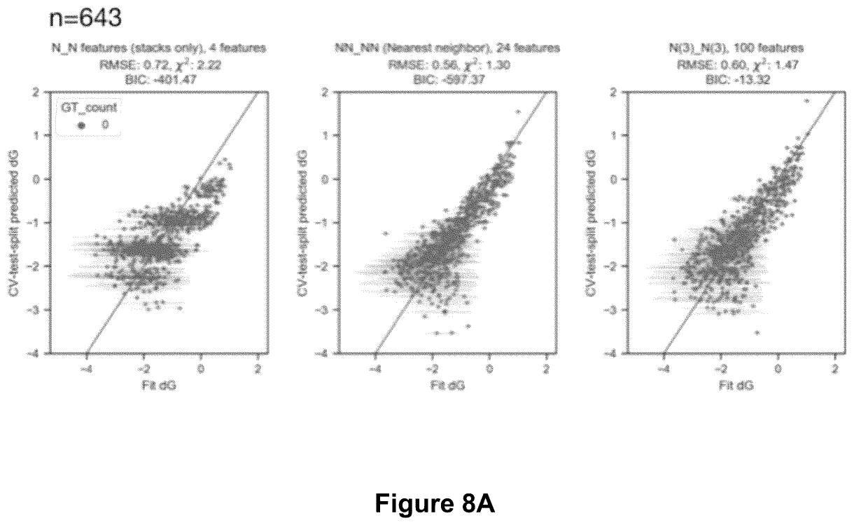

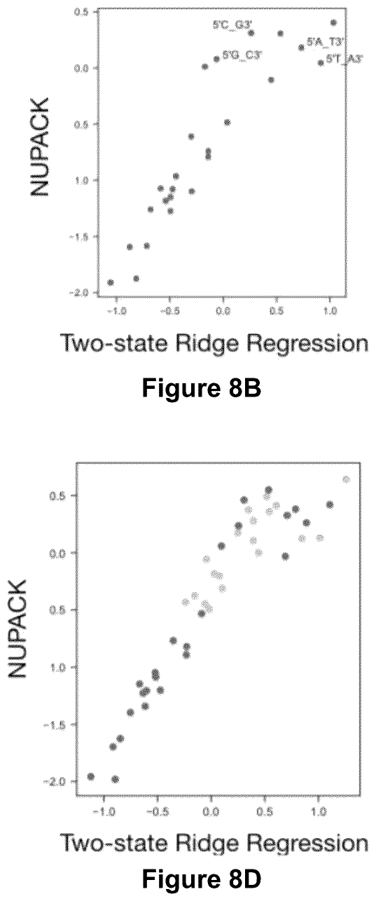

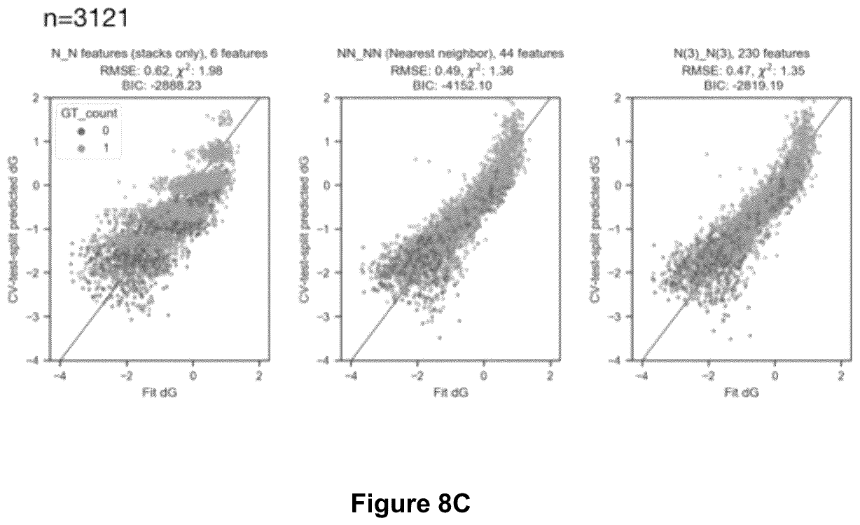

To test the nearest-neighbor model, linear regressions were fit to held-out subsets of the Watson-crick library and evaluated predictive power on the held-out subset. The base-pair, nearest-neighbor, and triplet-neighbor model resulted in test set RMSE values of 0.72, 0.56, and 0.60, respectively, and Bayesian Information Criterion (BIC) values of −401, −597, and −13 (FIG. 7B). This indicates that for Watson-Crick stacking, the nearest-neighbor model demonstrated the best performance and model quality given its number of parameters, as measured by the BIC. When introducing constructs that contained G-T mismatches (FIG. 8C), the base-pairing, nearest neighbor, and triplet-neighbor model resulted in RMSE values of 0.62, 0.49, and 0.47, and BIC values of −2999, −4152, and −2819, indicating that the triplet-neighbor model had the best predictive power, though not the best BIC. Future work will include identifying precisely which triple-stack features can be added to a nearest-neighbor model to maximally improve model information quality.

Nearest-neighbor parameters derived in this method were compared to parameters derived analogously from linear fits to NUPACK dG (37° C.) values, calculated using the SantaLucia 1998 parameters (FIG. 7C) and found that the resulting coefficients were in close agreement with the NUPACK parameters, with the largest deviations in closing base-pair parameters and parameters associated with G-T mismatches (orange, FIG. 8D). The differences in closing basepair parameters could be an artefact of the MANIfold hairpin system; the hairpin does not include a terminal stack, but instead a hairpin followed by two A-A mismatches, and a Cy3-BHQ fluor-quench pair, which also stabilizes the system.

It was desired to devise an inference system that did not rely on a two-state assumption to fit thermodynamic parameters. Ensemble-based inference systems have been demonstrated to result in superior models in the context of fitting duplex data as well as protein-binding data for inferring RNA parameters. The EternaFold inference system was extended to train a set of parameters (see Methods) by training a set of parameters that minimizes the difference between the experimentally-measured frac (quenched) at a given temperature and the calculated p (closing base pair). This does not assume that the entire hairpin is folded for the closing base pair to be folded and the fluorescent signal to be quenched. This inference system can also train a model based on a two-state system by fitting p (hairpin) instead of p (closing base pair). In initial tests training this system with the Watson-Crick+G-T mismatch data discussed above, it was found that parameters derived using an ensemble-aware method—fitting p (closing base pair)—and a two state model,—fitting p (hairpin)—resulted in discrepancies in the derived parameters, particularly G-T mismatch parameters. This indicates that this ensemble-aware method will be important for future use of this method in inferring parameters.

### Example 2: Develop and Deploy Molecular Engineering Platform for Functional RNA

Background: Methods of modeling RNA secondary structure generally draw from biochemical thermal melting of hundreds of different RNA structures, each laboriously collected for each sequence variant studied, to generate energetic contributions of nearest-neighbor bases. These rules form the foundations for current understanding of energetic stabilities of simple RNA structures. However, nearest-neighbor rules, which are so powerful for quantifying the energies associated with simple double-stranded structures, are derived from relatively limited thermodynamic datasets (hundreds of measurements) that have significant shortcomings. The diverse intramolecular interactions that determine the stability and three-dimensional structure of ssDNA and ssRNA-including, mismatched base bulges, stem loops, pseudo knots, g-quartets, divalent cation interactions, and non-canonical base pairs—are exponentially richer when compared to simple dsDNA thermodynamics, and have not been comprehensively quantitated. Thus, because the combinatorial space covered by DNA and RNA sequence is astronomical, high-throughput methods for quantitative biochemical investigations of RNA and DNA are necessary to quantitatively ground our understanding of stability and the determinants of structure and function.

Methods: Using methods described herein, the thermodynamic parameters of a massive array of DNA and RNA hairpins with variable regions in the stem were measured. These structures were generated from DNA oligos created using either error-prone oligonucleotide synthesis or array-based oligonucleotide synthesis (as previously described). All possible 4-11 base-pair stems with a constant tetra-base loop (˜16 million unique molecules) were generated for on-chip thermodynamic analysis by direct thermal melting measured with fluorescence-quenching readouts. After this first-order investigation is complete, all hairpins with all possible single base mismatches in hairpins less than 9 bases long can be investigated, as well as a subset of structures with hairpins 9 bases long and two stem mismatches. All possible 3-11 base loops with a constant stable stem (˜16 million unique molecules) can also be synthesized. Finally, all bulge loops of the form 2×1, 1×2, 2×2, 3×2, 2×3, 1×3, 3×1 and 3×3 in a defined 9 bp stem backbone can be synthesized. These measurements will generate a highly-multidimensional “periodic table” of DNA and RNA structure and thermodynamics parameters that might be easily transplanted into DNA and RNA structure prediction software, and multiply the current basis set of thermodynamic melt parameters by 3-4 orders of magnitude. In all cases, the calculated free energies can be compared with the high-throughput methods to a handful of DNA and RNA melting curves measured by UV-absorbance melting.

This example compared the measured energetic parameters of the stem loop structures with energies expected using standard nearest-neighbor methods. Then a new model for DNA and RNA stability by adding base-specific information regarding the context of mismatched bases, as well as non-canonical bases, still in nearest neighbor mode can be generated. These parameters can be obtained via linear regression similar to methods we previously used for RNA-protein interactions.

An energetic model can then be complicated by deriving best-fit energetic parameters for all possible overlapping three-base sequences (as opposed to two-base segments in nearest-neighbor model). A subset of data can be used to derive these parameters, and another subset can be used to assess the power of the model over a standard nearest-neighbors model. Four-base decomposition can also be explored to measure the relative power of each method to capture the observed variance in stabilities.

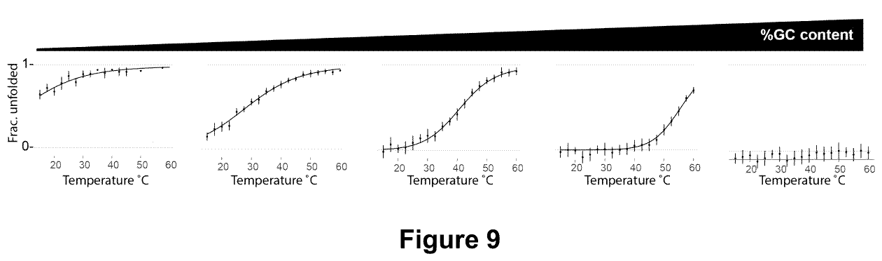

Results: Preliminary data illustrates quench-based thermal melting. Thermal melting of nucleic acid structures provides a straightforward means of assaying thermodynamic stability. To pilot melt-based measurements, a quenching based method for assaying thermodynamic stability of DNA was developed. Long (i.e. high-melting point, above 80° C.) labeled oligonucleotides were annealed to common regions engineered into the base of small hairpin structures to be investigated, allowing the generation of quenching-based signal dependent on hairpin structure. These hairpins were then perturbed with increasing temperature to generate a melting curve to determine the entropic and enthalpic contributions of free energy of unfolding. Preliminary data on DNA structures demonstrates that a quenching signal can be obtained on an instrument from labeled oligos at the base of a hairpin are illustrated in FIG. 9. Specifically, sample melt curves obtained from a variety of simple Watson-Crick stems of length 6 and increasing levels of GC content.

### Example 3: Engineer a “Chemical Nose” on a Sequencing Chip for Multifactorial, Near-Instantaneous Quantification of Small Molecules

Background: Molecular detection, characterization, and quantification are at the heart of diverse molecular diagnostics. Methods for quantifying or identifying small molecules span a continuum of techniques from ultra-general and complex methods such as mass spectrometry or nuclear magnetic resonance, to ultra-targeted and often simple techniques such as enzyme linked immunosorbent assays (ELISAs). However, general purpose methods for molecular identification of diverse molecules tend to be laborious and costly, whereas highly specific assays are often inexpensive, but provide only a limited window into chemical diversity present in a sample. To bridge the gap between expensive, general-purpose techniques and inexpensive and highly targeted molecular detection methods, many have suggested a paradigm inspired by the principle of olfaction, wherein many affinity-based, cross reactive sensors might be multiplexed to detect or quantify diverse biomolecules. This “molecular nose” approach has been used with some success to detect moderate number of proteins using tens of aptamer molecules. However, for the sensing of small molecules, a number of challenges to this paradigm have become evident. First, the quantitative quantification of molecules with highly similar molecular structures, or enantiomers, or absolute quantification of mixtures, have each proven challenging. Recent theoretical analysis has suggested that quantification of complex mixtures requires radically larger numbers of molecular sensors than have previously been employed. This example aims to demonstrate the utility of large-scale nucleic acid aptamer “chemical noses” for detecting related small molecules of diagnostic interest.

Methods: This embodiment focuses on training a chemical nose sensor across 20 different small molecules. This embodiment also aims to carry out more measurements with different starting “mother” aptamers with distinct sequences, likewise generating tens of thousands of variants from each of these starting points, aiming for aptamers that will have different chemical sensitivities to this collection of similar molecules. One aim is an array of ˜50,000-500,000 sensors capable of measuring arbitrary combinations of 20 related bile acid and steroid compounds with better than 20% error. Computational strategies aiming to link fluorescence signals beyond our initial linear biophysical model to recently-developed machine-learning models were expanded. Finally, the platform was expanded to different collections of molecules, including ATP, nucleosides, and related derivatives, as well as organophosphate compounds. Expanding this chemical nose platform to these diverse, and diagnostically relevant, compounds will clearly delineate the areas of useful application, and highlight the specific chemical differences that are challenging or more straightforward to differentiate. Another aim is to use multiple diverse aptamers as starting points for mutagenesis and generation of our aptamer arrays targeting these new classes of compounds, in order to span the widest possible chemical space.

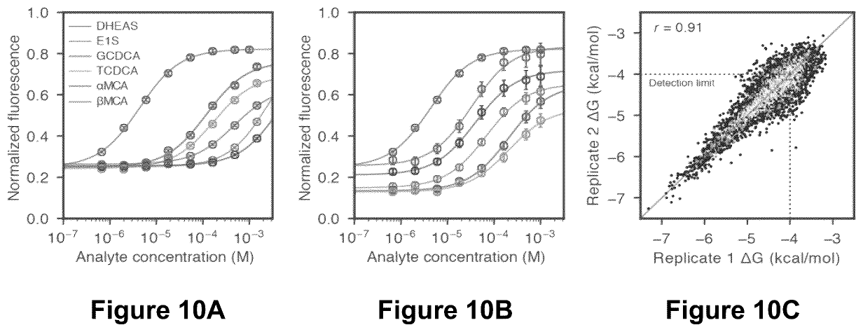

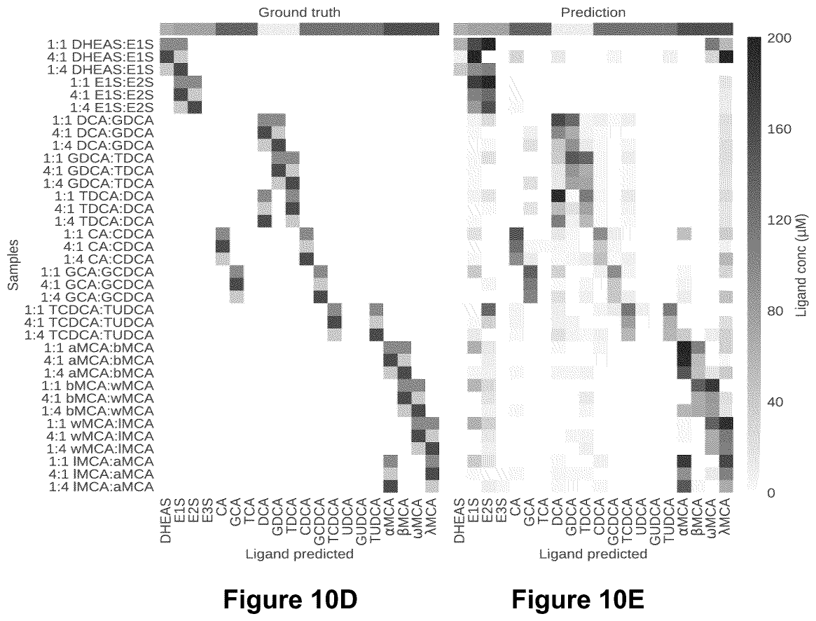

Results: FIG. 10A illustrates the observed fluorescence of a single aptamer sequence to diverse small molecule ligands illustrating differential sensitivity. FIG. 10B illustrates fluorescence curves for 6 distinct aptamers binding to a single ligand (DHEAS). FIG. 10C illustrates the reproducibility of observed delta G for binding across replicate experiments. Finally, FIGS. 10D-10E illustrate the quantification of true (FIG. 10D) and predicted estimates (FIG. 10E) of relatively simple mixtures of bile acids.

### Example 4: Performance of DNA Thermodynamics Models on Validation Set

Background: Machine learning models can allow for prediction of nucleic acid structure and/or thermodynamics. This embodiment tests performance of various machine learning models.

Methods: Models include 1) a k nearest neighbor model (k-NN) with k=8, distance between variants are the sum of string edit distance of DNA sequence and secondary structure dot bracket representation; 2) an ordinary least squares (OLS) model with 1331 features, which equivalent to the traditional nearest neighbor model; 3) a graph attention neural network with 3 graph convolution layers, a pooling layer and 2 linear layers; and 4) a TransformerConv, graph transformer network with 4 graph convolution layers, a pooling layer and 2 linear layers.

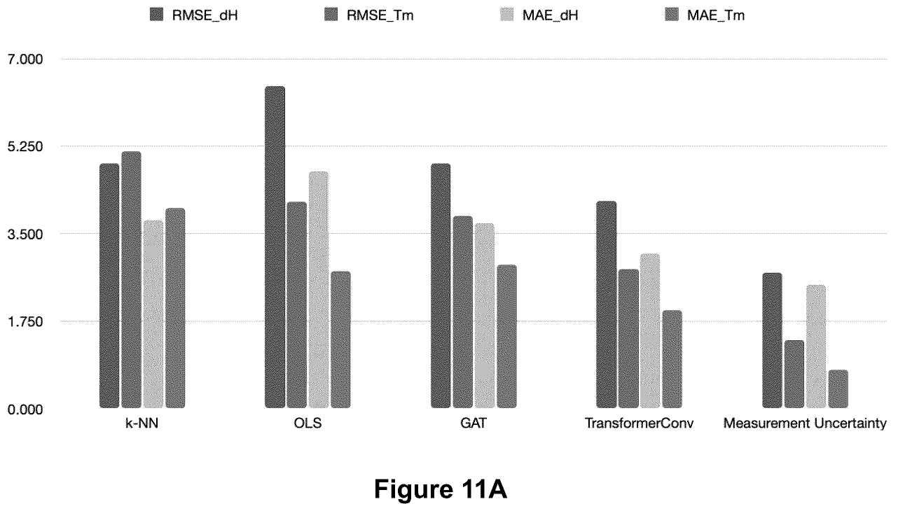

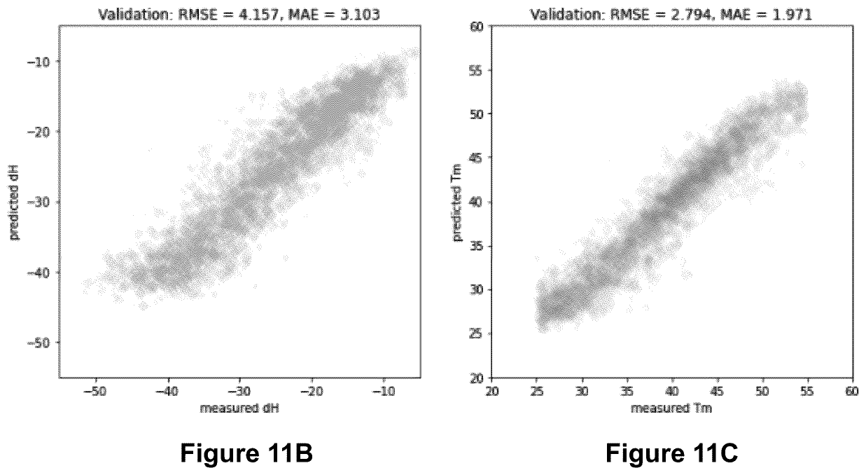

Results: FIG. 11A provides a bar chart of model prediction errors on the validation dataset. All models predict 2 parameters, dH and Tₘ of each DNA sequence with secondary structure information. Measurement uncertainty, bootstrapping estimate of measurement error from the data; RMSE, root mean square error; MAE, mean absolute error. FIGS. 11B-11C provide prediction of graph transformer network compared to experimental measurements on a validation set.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A method for measuring nucleic acid thermodynamics, comprising:
obtaining a library of nucleic acid molecules, wherein each molecule in the library comprises a first oligonucleotide complementary region, a second oligonucleotide complementary region, and a query region, wherein the query region comprises a sequence of interest to calculate thermodynamics of a secondary structure formed within the query region, wherein the first oligonucleotide complementary region is located 5′ of the query region and the second oligonucleotide complementary region is located 3′ of the query region;
affixing the library of nucleic acid molecules to a nucleic acid sequencing chip;
hybridizing a first oligonucleotide to the first oligonucleotide complementary region and a second oligonucleotide to the second oligonucleotide complementary region of each molecule in the library of nucleic acid molecules affixed to the sequencing chip, wherein the first oligonucleotide comprises a first tag at its 5′ end and the second oligonucleotide comprises a second tag at its 3′ end, wherein the first tag and the second tag are capable of interacting when within a specified distance each other, and wherein a structure formed in the query region brings the first tag and the second tag within the specified distance;
altering a parameter of the nucleic acid sequencing chip, wherein a change in the parameter affects a structure formed in the query region; and
measuring a signal emitted from at least one of the first tag and the second tag as the parameter changes.

2. The method of claim 1, wherein the parameter is selected from pH, salt composition, salt concentration, buffer composition, buffer concentration, organic molecule composition, organic molecule concentration, temperature, and combinations thereof.

3. The method of claim 1, wherein the parameter is salt composition.

4. The method of claim 3, wherein the salt within the salt composition is selected from the group consisting of: sodium chloride and potassium chloride.

5. The method of claim 1, wherein the parameter is buffer composition.

6. The method of claim 5, wherein the buffer within the buffer composition is selected from the group consisting of: sodium phosphate, sodium bisphosphate, sodium carbonate, sodium bicarbonate, potassium phosphate, potassium bisphosphate, potassium carbonate, potassium bicarbonate, sodium acetate, and potassium acetate.

7. The method of claim 1, wherein the parameter is temperature.

8. The method of claim 7, wherein the temperature ramps from approximately 4° C. to 90° C.

9. The method of claim 1, wherein the first tag or the second tag is a fluorophore.

10. The method of claim 1, wherein the first tag and the second tag are fluorophores.

11. The method of claim 10, wherein the emission wavelength of the first tag is the excitation wavelength of the second tag.

12. The method of claim 10, wherein the emission wavelength of the second tag is the excitation wavelength of the first tag.

13. The method of claim 1, wherein the first tag is a fluorophore and the second tag is a quencher.

14. The method of claim 13, wherein the emission wavelength of the first tag is an absorbance wavelength of the second tag.

15. The method of claim 1, wherein the first tag is a quencher and the second tag is a fluorophore.

16. The method of claim 15, wherein the emission wavelength of the second tag is an absorbance wavelength of the first tag.

17. The method of claim 1, wherein the sequencing chip is an Illumina flow cell.

18. The method of claim 1, further comprising sequencing each molecule in the affixed library of nucleic acid molecules.

19. The method of claim 18, wherein sequencing identifies a coordinate of each molecule in the affixed library of nucleic acid molecules.

20. The method of claim 1, further comprising transcribing each molecule in the affixed library of nucleic acid molecules into RNA, wherein hybridizing a first oligonucleotide hybridizes the first oligonucleotide to the RNA.

21. The method of claim 20, wherein measuring the signal comprises:
imaging the sequencing chip;
increasing temperature on the sequencing chip; and
reimaging the sequencing chip.

22. The method of claim 21, wherein measuring the signal further comprises:
reincreasing temperature on the sequencing chip; and
reimaging the sequencing chip.

23. The method of claim 1, wherein measuring the signal comprises:
imaging the sequencing chip;
increasing temperature on the sequencing chip; and
reimaging the sequencing chip.

24. The method of claim 23, wherein measuring the signal further comprises:
reincreasing temperature on the sequencing chip; and
reimaging the sequencing chip.

25. The method of claim 1, wherein the nucleic acid molecules are selected from DNA, RNA, LNA, and combinations thereof.

26. A method for predicting nucleic acid thermodynamics, comprising:
obtaining high-throughput measurements of nucleic acid thermodynamics;
training a machine learning model based on the thermodynamics of specific sequences in the high-throughput measurements; and
predicting thermodynamics of a query sequencing using the machine learning model.

27. The method of claim 26, wherein obtaining high-throughput measurements comprises:
obtaining a library of nucleic acid molecules, wherein each molecule in the library comprises a first oligonucleotide complementary region, a second oligonucleotide complementary region, and a query region, wherein the query region comprises a sequence of interest to calculate thermodynamics of a secondary structure formed within the query region, wherein the first oligonucleotide complementary region is located 5′ of the query region and the second oligonucleotide complementary region is located 3′ of the query region;
affixing the library of nucleic acid molecules to a nucleic acid sequencing chip;
hybridizing a first oligonucleotide to the first oligonucleotide complementary region and a second oligonucleotide to the second oligonucleotide complementary region of each molecule in the library of nucleic acid molecules affixed to the sequencing chip, wherein the first oligonucleotide comprises a first tag at its 5′ end and the second oligonucleotide comprises a second tag at its 3′ end, wherein the first tag and the second tag are capable of interacting when within a specified distance each other, and wherein a structure formed in the query region brings the first tag and the second tag within the specified distance;
altering a parameter of the nucleic acid sequencing chip, wherein a change in the parameter affects a structure formed in the query region; and
measuring a signal emitted from at least one of the first tag and the second tag as the parameter changes.

28. The method of claim 27, wherein the parameter is selected from pH, salt composition, salt concentration, buffer composition, buffer concentration, organic molecule composition, organic molecule concentration, temperature, and combinations thereof.

29. The method of claim 27, wherein the parameter is salt composition.

30. The method of claim 29, wherein the salt within the salt composition is selected from the group consisting of: sodium chloride and potassium chloride.

31. The method of claim 27, wherein the parameter is buffer composition.

32. The method of claim 31, wherein the buffer within the buffer composition is selected from the group consisting of: sodium phosphate, sodium bisphosphate, sodium carbonate, sodium bicarbonate, potassium phosphate, potassium bisphosphate, potassium carbonate, potassium bicarbonate, sodium acetate, and potassium acetate.

33. The method of claim 27, wherein the parameter is temperature.

34. The method of claim 33, wherein the temperature ramps from approximately 4° C. to 90° C.

35. The method of claim 27, wherein the first tag or the second tag is a fluorophore.

36. The method of claim 27, wherein the first tag and the second tag are fluorophores.

37. The method of claim 36, wherein the emission wavelength of the first tag is the excitation wavelength of the second tag.

38. The method of claim 36, wherein the emission wavelength of the second tag is the excitation wavelength of the first tag.

39. The method of claim 27, wherein the first tag is a fluorophore and the second tag is a quencher.

40. The method of claim 39, wherein the emission wavelength of the first tag is an absorbance wavelength of the second tag.

41. The method of claim 27, wherein the first tag is a quencher and the second tag is a fluorophore.

42. The method of claim 41, wherein the emission wavelength of the second tag is an absorbance wavelength of the first tag.

43. The method of claim 27, wherein the sequencing chip is an Illumina flow cell.

44. The method of claim 27, further comprising sequencing each molecule in the affixed library of nucleic acid molecules.

45. The method of claim 44, wherein sequencing identifies a coordinate of each molecule in the affixed library of nucleic acid molecules.

46. The method of claim 27, further comprising transcribing each molecule in the affixed library of nucleic acid molecules into RNA, wherein hybridizing a first oligonucleotide hybridizes the first oligonucleotide to the RNA.

47. The method of claim 46, wherein measuring the signal comprises:
imaging the sequencing chip;
increasing temperature on the sequencing chip; and
reimaging the sequencing chip.

48. The method of claim 47, wherein measuring the signal further comprises:
reincreasing temperature on the sequencing chip; and
reimaging the sequencing chip.

49. The method of claim 27, wherein measuring the signal comprises:
imaging the sequencing chip;
increasing temperature on the sequencing chip; and
reimaging the sequencing chip.

50. The method of claim 49, wherein measuring the signal further comprises:
reincreasing temperature on the sequencing chip; and
reimaging the sequencing chip.

51. The method of claim 27, wherein the nucleic acid molecules are selected from DNA, RNA, LNA, and combinations thereof.

52. A method for measuring interactions between a nucleic acid and another molecule comprising:
obtaining a library of nucleic acid molecules, wherein each molecule in the library comprises a query region, wherein the query region comprises a sequence of interest to determine an interaction between the query region and another molecule and a first tag affixed to the query region;
affixing the library of nucleic acid molecules to a nucleic acid sequencing chip;
introducing a query molecule to the nucleic acid sequencing chip to allow an interaction to form between the query region of at least one nucleic acid molecule in the library of nucleic acid molecules and the query molecule, wherein the query molecule comprises a second tag, and wherein an interaction between the query region of the at least one nucleic acid molecule and the query molecule brings the first tag and the second tag within a specified distance of each other, wherein the specified distance allows the first tag and second tag to interact;
altering a parameter of the nucleic acid sequencing chip, wherein a change in the parameter affects an interaction between a query region and a query molecule; and
measuring a signal emitted from at least one of the first tag and the second tag as the parameter changes.

53. The method of claim 52, wherein the parameter is selected from pH, salt composition, salt concentration, buffer composition, buffer concentration, organic molecule composition, organic molecule concentration, temperature, and combinations thereof.

54. The method of claim 52, wherein the parameter is salt composition.

55. The method of claim 54, wherein the salt within the salt composition is selected from the group consisting of: sodium chloride and potassium chloride.

56. The method of claim 52, wherein the parameter is buffer composition.

57. The method of claim 56, wherein the buffer within the buffer composition is selected from the group consisting of: sodium phosphate, sodium bisphosphate, sodium carbonate, sodium bicarbonate, potassium phosphate, potassium bisphosphate, potassium carbonate, potassium bicarbonate, sodium acetate, and potassium acetate.

58. The method of claim 52, wherein the parameter is temperature.

59. The method of claim 58, wherein the temperature ramps from approximately 4° C. to 90° C.

60. The method of claim 52, wherein the first tag or the second tag is a fluorophore.

61. The method of claim 52, wherein the first tag and the second tag are fluorophores.

62. The method of claim 61, wherein the emission wavelength of the first tag is the excitation wavelength of the second tag.

63. The method of claim 61, wherein the emission wavelength of the second tag is the excitation wavelength of the first tag.

64. The method of claim 52, wherein the first tag is a fluorophore and the second tag is a quencher.

65. The method of claim 64, wherein the emission wavelength of the first tag is an absorbance wavelength of the second tag.

66. The method of claim 52, wherein the first tag is a quencher and the second tag is a fluorophore.

67. The method of claim 66, wherein the emission wavelength of the second tag is an absorbance wavelength of the first tag.

68. The method of claim 52, wherein the sequencing chip is an Illumina flow cell.

69. The method of claim 52, further comprising sequencing each molecule in the affixed library of nucleic acid molecules.

70. The method of claim 69, wherein sequencing identifies a coordinate of each molecule in the affixed library of nucleic acid molecules.

71. The method of claim 52, further comprising transcribing each molecule in the affixed library of nucleic acid molecules into RNA, wherein hybridizing a first oligonucleotide hybridizes the first oligonucleotide to the RNA.

72. The method of claim 71, wherein measuring the signal comprises:
imaging the sequencing chip;
increasing temperature on the sequencing chip; and
reimaging the sequencing chip.

73. The method of claim 72, wherein measuring the signal further comprises:
reincreasing temperature on the sequencing chip; and
reimaging the sequencing chip.

74. The method of claim 52, wherein measuring the signal comprises:
imaging the sequencing chip;
increasing temperature on the sequencing chip; and
reimaging the sequencing chip.

75. The method of claim 74, wherein measuring the signal further comprises:
reincreasing temperature on the sequencing chip; and
reimaging the sequencing chip.

76. The method of claim 52, wherein the nucleic acid molecules are selected from DNA, RNA, LNA, and combinations thereof.

77. The method of claim 52, wherein the query molecule is selected from the group consisting of: a nucleic acid, a protein, a peptide, a carbohydrate, an organic compound, and combinations thereof.

78. A method for determining composition of a complex mixture, comprising:
obtaining a library of nucleic acid molecules affixed to a sequencing chip, wherein each molecule in the library comprises an aptamer region, a self-complementary region, a first complementary region, and a second complementary region, wherein the aptamer region is flanked by the self-complementary region and the second complementary region, and the first complementary region is located adjacent to the second complementary region, and wherein the self-complementary region is complementary to the second complementary region;
hybridizing a first oligonucleotide to the first complementary region and a second oligonucleotide to the second complementary region of each molecule in the library of nucleic acid molecules, wherein the first oligonucleotide comprises a first tag and the second oligonucleotide comprises a second tag, wherein the first tag and the second tag are capable of interacting when within a specified distance each other, and wherein hybridization of the first oligonucleotide to the first complementary region and the second oligonucleotide to the second complementary region brings the first tag and second tag within the specified distance;
introducing a sample to the sequencing chip, wherein the sample comprises small molecules of interest, wherein an interaction between a small molecule in the sample to an aptamer region causes a conformational change in a nucleic acid molecule which displaces the second oligonucleotide from the second complementary region and allows the self-complementary region to bind to the second complementary region; and
measuring a signal emitted from the first tag as an indicator of an interaction between an aptamer region and a small molecule interaction.

79. The method of claim 78, wherein the sample is selected from a biological sample and an environmental sample.

80. The method of claim 78, wherein the first tag or the second tag is a fluorophore.

81. The method of claim 78, wherein the first tag and the second tag are fluorophores.

82. The method of claim 81, wherein the emission wavelength of the first tag is the excitation wavelength of the second tag.

83. The method of claim 81, wherein the emission wavelength of the second tag is the excitation wavelength of the first tag.

84. The method of claim 78, wherein the first tag is a fluorophore and the second tag is a quencher.

85. The method of claim 84, wherein the emission wavelength of the first tag is an absorbance wavelength of the second tag.

86. The method of claim 78, wherein the first tag is a quencher and the second tag is a fluorophore.

87. The method of claim 86, wherein the emission wavelength of the second tag is an absorbance wavelength of the first tag.

88. The method of claim 78, wherein the sequencing chip is an Illumina flow cell.

89. The method of claim 78, further comprising sequencing each molecule in the affixed library of nucleic acid molecules.

90. The method of claim 89, wherein sequencing identifies a coordinate of each molecule in the affixed library of nucleic acid molecules.

91. The method of claim 78, wherein the nucleic acid molecules are selected from DNA, RNA, LNA, and combinations thereof.


SEQUENCE LISTING (9 sequences)

SEQ ID NO: 1 - DNA, 17 bp, synthetic construct
  source 1..17: mol_type = other DNA
gactannnnnnntagtc

SEQ ID NO: 2 - DNA, 18 bp, synthetic construct
  source 1..18: mol_type = other DNA
gactannnnnnnntagtc

SEQ ID NO: 3 - DNA, 22 bp, synthetic construct
  source 1..22: mol_type = other DNA
gcgnnnccggaaacggnnncgc

SEQ ID NO: 4 - DNA, 21 bp, synthetic construct
  source 1..21: mol_type = other DNA
gcgnnccggaaacggnnncgc

SEQ ID NO: 5 - DNA, 21 bp, synthetic construct
  source 1..21: mol_type = other DNA
gcgnnnccggaaacggnncgc

SEQ ID NO: 6 - DNA, 22 bp, synthetic construct
  source 1..22: mol_type = other DNA
gcgnnnnccggaaacggnncgc

SEQ ID NO: 7 - DNA, 22 bp, synthetic construct
  source 1..22: mol_type = other DNA
gcgnnccggaaacggnnnncgc

SEQ ID NO: 8-9 - skipped (2 entries)